(19) United States
(12) Patent Application Publication
(10) Pub. No.: US 2022/0307063 A1
(43) Pub. Date: Sep. 29, 2022

(54) METHOD FOR ANALYZING MICROBIAL FLORA

(71) Applicant: NATIONAL INSTITUTE OF ADVANCED INDUSTRIAL SCIENCE AND TECHNOLOGY, Tokyo (JP)

(72) Inventors: Shunsuke TOMITA, Tsukuba-shi, Ibaraki (JP); Ryoji KURITA, Tsukuba-shi, Ibaraki (JP); Hideyuki TAMAKI, Tsukuba-shi, Ibaraki (JP); Hiroyuki KUSADA, Tsukuba-shi, Ibaraki (JP); Naoshi KOJIMA, Tsukuba-shi, Ibaraki (JP); Sayaka ISHIHARA, Tsukuba-shi, Ibaraki (JP); Koyomi MIYAZAKI, Tsukuba-shi, Ibaraki (JP); Isao YUMOTO, Tsukuba-shi, Ibaraki (JP)

(21) Appl. No.: 17/615,853

(22) PCT Filed: Jun. 24, 2020
(86) PCT No.: PCT/JP2020/024687

§ 371 Date: Dec. 2, 2021

(30) Foreign Application Priority Data
Jun. 26, 2019 (JP) 2019-118640

(51) Int. Cl. C12Q 1/04; G01N 21/64

(52) U.S. Cl. CPC C12Q 1/04; G01N 21/64

(57) ABSTRACT

A method for analyzing a microbiota, comprising (1) dissolving a probe capable of non-specifically interacting with a plurality of microorganisms in a plurality of solvents having different ionic strengths and pH levels, wherein the probe comprises: (a) a cationic polymer and (b) an environment-sensitive fluorophore; (2) adding a test sample containing the microbiota to a plurality of probe solutions prepared in the step (1), thereby microorganisms in the test sample and the probe are interacted non-specifically; (3) measuring fluorescence intensities of the plurality of probe solutions to which the test sample has been added in the step (2); and (4) comparing the pattern of fluorescence intensities obtained in the step (3) with the pattern of fluorescence intensities obtained from a reference sample.

### TECHNICAL FIELD

The present invention relates to a method for determining the types and/or amounts of microorganisms contained in microbiota and a method for determining the states and characteristics of individual animals from which microbiota are isolated.

### BACKGROUND ART

At least 100 trillion microorganisms such as bacteria, fungi, and viruses naturally inhabit the human body. They colonize and live while maintaining populations unique to parts such as the skin, the mouth, and the alimentary canal, and such microbial groups are collectively called indigenous microbiota. It is known that indigenous microbiota interact with hosts closely and have various effects on the hosts. It has been found in recent years that indigenous microbiota, especially gut microbiota (intestinal florae), are strongly related to the health conditions and disease conditions of hosts. It has been pointed out that, in patients with various diseases such as obesity, type 2 diabetes, and allergies, or are on the autism spectrum disorder, abnormalities in the compositions of gut microbiota and a decrease in diversity of gut microbiota (dysbiosis) are seen (Non-Patent Documents 1 and 2). Therefore, the understanding and control of gut microbiota is being focused on from the viewpoint of health care, or the prevention or the treatment of diseases (Non-Patent Document 2).

Indigenous microbiota are presently analyzed by phylogenetic systematics based on the sequences of bacterial 16S ribosomal RNAs (16S rRNA analysis), or whole genome shotgun metagenome analysis, and all of these comprehensively identify the types of microorganisms constituting microbiota. However, 16S rRNA analysis has a problem in that it cannot be ruled out that there may be present unknown microorganisms that cannot be amplified by PCR, or even if all of the types of microorganisms can be identified, it is difficult to analyze them quantitatively. Whole genome shotgun metagenome analysis is much more accurate than 16S rRNA analysis; however, it is laborious, time-consuming, and expensive, due to analysis of all the genomes, and it is therefore not practical on a routine basis.

A method for analyzing microbiota based on cross-reactive sensing is an example of a method that may solve the abovementioned problems. In this case, microorganisms constituting microbiota are not identified comprehensively, and the compositions of microbiota are distinguished by analyzing the sum of the non-specific interactive reactions between various microorganisms and cross-reactive molecules statistically using molecules that various target microorganisms contained in microbiota that are analytical targets of cross-reaction. Thus, analytic methods based on cross-reactive sensing have the advantage that they do not require the development of individual molecules that interact specifically with a particular microorganism.

It has been recently reported that a mixed sample of two or three types of bacteria can be distinguished using cross-reactive sensing (Non-Patent Documents 3 and 4). However, a microbiota comprises several hundred or more types of microorganisms, and a probe and a method for discriminating a mixed sample of such a wide and vast variety of types of microorganisms by cross-reactive sensing have not yet been reported.

### CITATION LIST

### Non-Patent Documents

Non-Patent Document 1: Nat. Microbiol., 2018, Vol. 3, pp. 8-16
Non-Patent Document 2: Nature, 2016, Vol. 535, pp. 94-103
Non-Patent Document 3: Adv. Healthcare Mater., 2018, Vol. 7, Article No. 1701370
Non-Patent Document 4: Anal. Chem., 2017, Vol. 89, pp. 3208-3216

### SUMMARY OF INVENTION

### Technical Problem

The present invention has been made in order to provide a method that solves the problems in the prior art and distinguishes microbiota derived from animals easily and with high accuracy.

### Solution to Problem

The present inventors have succeeded in distinguishing a wide variety of types of proteins and types and/or amounts of posttranslational modifications by cross-reactive sensing (WO2018/088510). The present inventors have established a method that can distinguish the states and/or characteristics of microbiota and the animals from which they were derived with high accuracy based on the abovementioned method.

That is, according to one embodiment, the present invention provides a method for analyzing a microbiota, comprising (1) dissolving a probe capable of non-specifically interacting with a plurality of microorganisms in a plurality of solvents having different ionic strengths and pH levels, wherein the probe comprises: (a) a cationic polymer comprising at least five primary amino groups in one molecule and having a weight-average molecular weight of 1,000 to 500,000 and (b) an environment-sensitive fluorophore, wherein the fluorophore is covalently bonded to some of the primary amino groups in the cationic polymer; (2) adding a test sample containing the microbiota to a plurality of probe solutions prepared in the step (1), thereby microorganisms in the test sample and the probe are interacted non-specifically; (3) measuring fluorescence intensities of the plurality of probe solutions to which the test sample has been added in the step (2); and (4) comparing the pattern of fluorescence intensities obtained in the step (3) with the pattern of fluorescence intensities obtained from a reference sample, and thereby the types and/or amounts of the microorganisms constituting the microbiota are determined.

The environment-sensitive fluorophore is preferably an aggregation-induced emission fluorophore, and is, for example, preferably tetraphenylethylene or a derivative thereof.

The cationic polymer is preferably a linear or branched polyamino acid, polyallylamine, polyamidoamine, or polyalkyleneimine.

The polyamino acid is preferably polylysine or polyornithine.

The environment-sensitive fluorophore is covalently bonded to preferably 1 to 50% of the primary amino groups in the cationic polymer.

A functional group selected from the group consisting of a guanidium group, a carboxyl group, and an amino acid is preferably introduced into at least some of the primary amino groups not covalently bonded to the environment-sensitive fluorophore in the cationic polymer.

The measurement of the fluorescence intensities in the step (3) is preferably performed at a plurality of excitation wavelengths and emission wavelengths.

Types and/or amounts of microbial strains comprised in the microbiota are preferably determined by the step (4).

It is preferable that the microbiota be derived from an individual animal, and a state and/or a characteristic of the individual animal be determined by the step (4).

The individual animal is preferably a human.

### Advantageous Effects of Invention

According to the method related to the present invention, microorganisms constituting microbiota can be qualitatively and quantitatively analyzed easily and with high accuracy by dissolving only one or a few types of probes in solvents having different ionic strengths and/or pH levels for use without the need to comprehensively identify a wide and vast variety of types of the microorganisms constituting the microbiota based on the pattern of fluorescence intensities reflecting the sum of the non-specific interactions between various microorganisms constituting the microbiota and a probe. According to the method related to the present invention, for example, the state and/or the characteristic of an animal individual which is an analytical target and from which the microbiota is derived can therefore be determined easily only by comparing the patterns of fluorescence intensities using microbiota derived from animal individuals having different health conditions and constitutions as reference samples.

### BRIEF DESCRIPTION OF DRAWINGS

FIG. 1 is a diagram illustrating structural formulae of probes synthesized in Examples (probes 1 to 12). Numerical values in parentheses show the C log P values of sites into which functional groups are introduced, respectively.

FIG. 2 is diagram illustrating the results of characterization for the probe 1 (-None) and the probe 5 (-Nle).

FIG. 3 is the heat map of fluorescence intensities (I) obtained by 16 types of bacteria×2 solvent conditions×12 probes×2 wavelength sets×11 measurements.

FIG. 4 is a graph obtained by analyzing the results of FIG. 3 by linear discriminant analysis and plotting the resulting quadratic discriminant scores.

FIG. 5 is a graph obtained by analyzing change in fluorescence intensity (I−I₀) obtained by 16 types of bacteria×2 solvent conditions×12 probes×2 wavelength sets×11 measurements by linear discriminant analysis and plotting the resulting quadratic discriminant scores.

FIG. 6 is a graph obtained by changing the labels of the bacteria into phyla in the results of FIG. 3 for analysis by linear discriminant analysis and plotting the resulting quadratic discriminant scores.

FIG. 7 is a graph showing the compositions of microbiota samples in obesity models.

FIG. 8 is the heat map of fluorescence intensities (I) obtained by 4 microbiota samples of obesity models×2 solvent conditions×6 probes×2 wavelength sets×22 measurements.

FIG. 9 is a graph obtained by analyzing the results of FIG. 8 by linear discriminant analysis and plotting the resulting quadratic discriminant scores.

FIG. 10 is typical actograms of mice of (a) a control group and (b) a sleep disorder model group.

FIG. 11 is the heat map of fluorescence intensities (I) obtained by 8 samples (4 control groups and 4 stress groups)×2 solvent conditions×6 probes×2 wavelength sets×22 measurements.

FIG. 12 is a graph obtained by analyzing the results of FIG. 11 by linear discriminant analysis and plotting the resulting quadratic discriminant scores.

FIG. 13 is a graph obtained by changing the labels of the samples depending on whether the mice were stressed or unstressed in the results of FIG. 12 for analysis by linear discriminant analysis and plotting the resulting linear discriminant scores.

FIG. 14 is a graph which illustrates change in the exercise quantity of mice.

FIG. 15 is the heat map of fluorescence intensities (I) obtained by 8 mouse microbiota samples×2 solvent conditions×6 probes×2 wavelength sets×6 measurements.

FIG. 16 is a graph obtained by analyzing the results of FIG. 15 by linear discriminant analysis and plotting the resulting quadratic discriminant scores.

FIG. 17 is a graph obtained by changing the labels of the samples depending on whether the running wheel was fixed or unfixed, analyzing the results of FIG. 16 by linear discriminant analysis, and plotting the resulting linear discriminant scores.

FIG. 18 is the heat map of fluorescence intensities (I−I₀) obtained by 8 types of E. coli×2 solvent conditions×6 probes×2 wavelength sets×11 measurements.

FIG. 19 is a graph obtained by analyzing the results of FIG. 18 by linear discriminant analysis and plotting the resulting quadratic discriminant scores.

FIG. 20 is a graph obtained by analyzing the results of FIG. 18 by linear discriminant analysis and plotting the resulting quadratic discriminant scores and cubic discriminant scores.

FIG. 21 is a diagram illustrating structural formulae of probes synthesized in Examples (probes 13 to 17). Numerical values in parentheses represent the C log P values of sites into which functional groups are introduced, respectively.

FIG. 22 is diagram illustrating the results of characterization for the probe 13 (-None/F) and the probe 14 (-Nle/F).

FIG. 23 is a figure showing the results obtained by dyeing cotton fabrics with fermented liquid after different elapsed days.

FIG. 24 is the heat map of the fluorescence intensities (I−I₀) obtained by 15 indigo dyeing bacterium flora samples×2 solvent condition×5 probes×3 wavelength sets×9 measurements.

FIG. 25 is a graph obtained by analyzing the results of FIG. 24 by linear discriminant analysis and plotting the resulting quadratic discriminant scores.

### DESCRIPTION OF EMBODIMENTS

Although the present invention will be described in detail hereinafter, the present invention is not limited to the embodiments described herein.

According to a first embodiment, the present invention is a method for analyzing a microbiota, comprising (1) dissolving a probe capable of non-specifically interacting with a plurality of microorganisms in a plurality of solvents having different ionic strengths and pH levels, wherein the probe comprises: (a) a cationic polymer comprising at least five primary amino groups in one molecule and having a weight-average molecular weight of 1,000 to 500,000 and (b) an environment-sensitive fluorophore, wherein the fluorophore is covalently bonded to some of the primary amino groups in the cationic polymer; (2) adding a test sample containing the microbiota to a plurality of probe solutions prepared in the step (1), thereby microorganisms in the test sample and the probe are interacted non-specifically; (3) measuring fluorescence intensities of the plurality of probe solutions to which the test sample has been added in the step (2); and (4) comparing the pattern of fluorescence intensities obtained in the step (3) with the pattern of fluorescence intensities obtained from a reference sample.

First, a probe used in the method of the present embodiment will be described. The probe used in the method of the present embodiment comprises (a) a cationic polymer comprising at least five primary amino groups in one molecule and having a weight-average molecular weight of 1,000 to 500,000 and (b) an environment-sensitive fluorophore, wherein the fluorophore is covalently bonded to some of the primary amino groups in the cationic polymer.

The cationic polymer (a) used in the probe of the present embodiment may be any polymer that has a weight-average molecular weight of 1,000 to 500,000 and comprises at least five primary amino groups in one polymer molecule. Here, the “polymer” means a compound prepared by polymerization of two or more monomers that may be the same or different, and may accordingly be a homopolymer or a copolymer. The polymer may have any degree of polymerization, and the “polymer” therefore includes an oligomer.

The cationic polymer according to the present embodiment has a weight-average molecular weight of 1,000 to 500,000, preferably 1,500 to 200,000, and particularly preferably 2,000 to 100,000.

The cationic polymer of the probe according to the present embodiment comprises at least five, preferably seven or more, and particularly preferably ten or more primary amino groups in one molecule of the polymer.

According to the present embodiment, the cationic polymer that can be used for the probe is preferably a linear or branched polyamino acid, polyallylamine, polyamidoamine, or polyalkyleneimine. Furthermore, these cationic polymers may be copolymers with polyethylene glycol.

According to the present embodiment, the polyamino acid that can be used for the probe may be a polymer of a single type of amino acid residues or may be a polymer of multiple different types of amino acid residues. In addition, the amino acid residues that constitute the polyamino acid may be in L-form or D-form. Examples of the polyamino acid include polylysine, polyornithine, a random copolymer of lysine and phenylalanine, and a random copolymer of lysine and tyrosine. The polyamino acid according to the present embodiment is preferably polylysine or polyornithine.

According to the present embodiment, examples of the polyalkyleneimine that can be used for the probe include polyethyleneimine, polypropyleneimine, and polybutyleneimine. The polyalkyleneimine according to the present embodiment is preferably polyethyleneimine.

As long as the environment-sensitive fluorophore (b) of the probe according to the present embodiment is a fluorophore that changes the fluorescence properties depending on the surrounding environment of the fluorescent molecule, the environment-sensitive fluorophore (b) may be any fluorophore. Examples of such a fluorophore include, but are not limited to, a fluorophore that changes fluorescence properties depending on the surrounding polarity of the fluorescence molecule, a fluorophore that changes fluorescence properties depending on the surrounding pH level of the fluorescence molecule, and a fluorophore that changes fluorescence properties depending on the surrounding congestion degree of the fluorescence molecule.

Examples of the fluorophore that changes fluorescence properties depending on the surrounding polarity of the fluorescence molecule include fluorophores having a naphthalene sulfonic acid structure, such as 5-dimethylaminonaphthalene-1-sulfonyl (dansyl), 1-anilinonaphthalene-8-sulfonic acid (ANS), N-methyl-2-anilinonaphthalene-6-sulfonic acid (MANS), and 2-p-toluidinylnaphthalene-6-sulfonic acid (TNS); fluorophores having a benzofurazan structure, such as 4-(N,N-dimethylaminosulfonyl)-2,1,3-benzoxadiazole (DBD), 7-nitro-2,1,3-benzoxadiazole (NBD), 4-(aminosulfonyl)-2,1,3-benzoxadiazole (ABD), and ammonium 2,1,3-benzoxadiazole-4-sulfate (SBD); or fluorescent derivatives thereof.

Examples of the fluorophore that changes fluorescence properties depending on the surrounding pH level of the fluorescence molecule include fluorophores having a xanthene structure, such as fluorescein, fluorescein isothiocyanate (FITC), 5(6)-carboxyfluorescein (5(6)-FAM), 2′-7′-bis(carboxyethyl)-5(6)-carboxyfluorescein (BCECF), and seminaphtharhodafluorescein (SNARF); fluorophores having a pyrene structure, such as trisodium 8-hydroxy pyrene-1,3,6-trisulfonate (HTPS); or fluorescent derivatives thereof.

Examples of the fluorophore that changes fluorescence properties depending on the surrounding congestion degree of the fluorescence molecule include aggregation-induced emission (AIE) fluorophores, such as tetraphenylethylene (TPE), 10,10′,11,11′-tetrahydro-5,5′-bidibenzo[a,d][7]annulenylidene (THBA) and 1,1,2,3,4,5-hexaphenylsilole (HPS); or fluorescent derivatives thereof. The environment-sensitive fluorophore which can be used for the probe according to the present embodiment is preferably an AIE fluorophore, and is particularly preferably TPE.

In the probe according to the present embodiment, the environment-sensitive fluorophore (b) is introduced into some of the primary amino groups in the cationic polymer (a) by covalent bonds. Here, “some” is preferably 1% to 50%, particularly preferably 5% to 20% of the primary amino groups in the cationic polymer molecule.

The probe according to the present embodiment can be synthesized by a known chemical synthesis method or a chemical synthesis method described in the following Examples or equivalent thereto.

In the probe according to the present embodiment, a functional group may be introduced into at least some of the primary amino groups into which the environment-sensitive fluorophore has not been introduced. The functional group may have a positive charge or a negative charge. The functional group selected from the group consisting of a guanidium group, a carboxyl group, and an amino acid can be used. Examples of the amino acid include leucine, valine, isoleucine, tyrosine, tryptophane, phenylalanine, serine, asparagine, glutamine, and derivatives thereof. One or more of the abovementioned functional groups can be optionally selected and introduced into the probe according to the present embodiment.

The introduction of a functional group into an amino group can be performed by a known chemical synthesis method. The introduction of a guanidium group into an amino group can be performed by, for example, using 1H-pyrazole-1-carboxamidine hydrochloride. The introduction of a carboxyl group can be performed by, for example, acetylation reaction using carboxylic anhydride, such as acetic anhydride, phthalic anhydride, and naphthalene dicarboxylic anhydride. The introduction of an amino acid into an amino group can be performed by, for example, dehydration condensation of the carboxyl group of the amino acid.

The probe according to the present embodiment can interact with a plurality of microorganisms non-specifically. The “microorganisms” that the probe targets may be any microorganism in any class, such as a bacterium, fungus, protozoan, or virus, and may be a mixture of microorganisms in a plurality of classes thereof. Microorganisms that the probe according to the present embodiment targets can include not only known microorganisms but also unknown microorganisms.

The microorganisms that the probe according to the present embodiment targets are preferably bacteria. The bacteria that the probe according to the present embodiment targets may be any class of bacteria, for example, may be either of gram-negative and gram-positive bacteria, may be either anaerobic and aerobic bacteria, or may be a mixture of a plurality of types of these bacteria. The probe according to the present embodiment can interact with either live bacteria or dead bacteria.

In the method of the present embodiment, the above-described probe is dissolved in a plurality of solvents having different ionic strengths and/or pH levels. Each solvent for dissolving the probe may be an aqueous solvent comprising any buffer and/or salt. Examples of the buffer include MES, MOPS, EPPS, HEPES, tris, phosphate, acetate, citrate, borate, and glycine buffers. Examples of the salt include NaCl, KCl, MgCl₂, Na₂SO₄, K₂SO₄, MgSO₄, NaI, and NaSCN. According to the present embodiment, the pH of the solvent is preferably 4.0 to 10.0 and is particularly preferably 5.0 to 7.0. In the present invention, the ionic strength of the solvent is preferably 10 to 500 mM. The concentration of the probe according to the present embodiment is preferably 0.1 to 100 μg/mL.

In the method of the present embodiment, the solvents having, for example, two or more, preferably three or more, particularly preferably six or more, different ionic strengths and/or pH conditions can be used. In addition, in the method of the present embodiment, one type of probe may be used, but many types of probes are more preferably used, for example, two or more, preferably three or more, and particularly preferably six or more types of probes can be used. For example, six types of probe solutions can be prepared using two types of solvents and three types of probes. As a result, six-dimensional data can be obtained from one test sample. For example, 15 types of probe solutions can be prepared using five types of solvents and three types of probes. As a result, 15-dimensional data can be obtained from one test sample. Thus, higher multidimensional data can be obtained by increasing the numbers of solvent types and probe types.

Subsequently, the test sample containing a microbiota is added to a plurality of probe solutions. Here, the “microbiota” means an aggregate of a plurality of microorganisms that exist in a certain specific environment. The microbiota can comprise, for example, at least 100 types, 300 types, 500 types, 700 types, 1,000 types, or more types of microorganisms. The microbiota can comprise various classes and/or types of microorganisms such as bacteria, fungi, protozoa, and viruses. The method of the present embodiment can target a microbiota comprising any classes and/or types of microorganisms. Examples of the environment in which the microbiota exists include the surface or the inside of an individual animal or plant, soil, seawater, and river water.

The method of the present embodiment can target any microbiota derived from any environment, and preferably targets a microbiota derived from an individual animal. An individual animal from which a microbiota is derived may be any individual vertebrate or any individual invertebrate, and is preferably an individual mammal such as a mouse, a rat, a rabbit, a dog, a nonhuman primate, or a human, and particularly preferably a human. The microbiota in an individual animal exists in the epithelium of a tissue (for example, the alimentary canal, the mouth, a nasal cavity, the skin, the respiratory organs, the genitals, or the like), and the microbiota analyzed in the method of the present embodiment may be a microbiota derived from any tissue epithelium. The microbiota derived from the animal individual and analyzed in the method of the present embodiment is preferably an oral microbiota (oral flora) or a gut microbiota (an intestinal microbiota) (intestinal flora), and is particularly preferably a gut microbiota.

The test sample containing the microbiota derived from the individual animal can be prepared by a known method. For example, a test sample containing an oral bacterial flora can be prepared from dental plaque, saliva, etc., and a test sample containing a gut microbiota can be prepared from feces. The final concentration of the microorganisms added to a probe solution may be, for example, OD₆₀₀=0.002 to 0.500 and preferably OD₆₀₀=0.010 to 0.100. When the concentration of the microorganisms in the test sample is unknown, the sample is suitably diluted gradually, and the diluted samples may be added to probe solutions.

In this step, the probe interacts non-specifically with the microorganisms contained in the microbiota regardless of the class and kind thereof. A human gut microbiota contains, for example, 1,000 or more types of bacteria belonging to phyla such as Bacteroidetes, Proteobacteria, Actinobacteria, and Firmicutes, and the probe can interact with all thereof non-specifically.

Subsequently, the fluorescence intensities of the plurality of probe solutions after adding the test sample are measured. In the method of the present embodiment, the fluorescence intensities can be measured at an excitation wavelength of 300 to 500 nm and an emission wavelength of 400 to 700 nm. In addition, in the method of the present embodiment, for each measured object, fluorescence intensities at a plurality of sets of excitation wavelength/emission wavelength (e.g., excitation wavelength (nm)/emission wavelength (nm): 330/480, 345/505, 360/530, etc.) are preferably measured. For example, fluorescence intensities at two sets, three sets, or four sets of excitation wavelength/emission wavelength can be measured.

The fluorescence intensities of the probe solutions change depending on the types and amounts of microorganisms contained in the added sample, and further depending on the conditions of the solvents dissolving the probe. Accordingly, a unique pattern of fluorescence intensities (fluorescent fingerprint) for the sample can be obtained by this step.

Subsequently, the pattern of fluorescence intensities obtained from the test sample is compared with the pattern of fluorescence intensities obtained from a reference sample. The pattern of fluorescence intensities of a reference sample may be obtained by measuring fluorescence intensities concurrently with those of a test sample, or a predetermined pattern of fluorescence intensities in advance may be used. The comparison of pattern of fluorescence intensities can be preferably performed by compressing the dimension number by multivariate analysis, such as principal component analysis, linear discriminant analysis, or hierarchical clustering analysis, and compressing and converting the difference between the fluorescent fingerprints into two-dimensions or three-dimensions.

The method of the present embodiment analyzes the microbiota only based on the difference in the pattern of fluorescence intensities. In a conventional method, the microbiota profile had to be obtained by comprehensively identifying the types of microorganisms constituting a microbiota by 16S rRNA analysis or whole genome shotgun metagenome analysis. Even if the types of microorganisms constituting the microbiota could be identified in that case, the comprehensive quantification thereof was difficult, and the identification of different strains of the same microbial species was also difficult. In the method of the present embodiment, the pattern of fluorescence intensities that reflects the sum of the non-specific interactions between various microorganisms that constitute the microbiota and the probe (fluorescent fingerprint) is obtained in the meantime, and qualitative and quantitative differences in the microorganisms that constitute the microbiota are detected by comparison with the pattern of fluorescence intensities of a reference sample.

Thus, in the method of the present embodiment, for example, a fluorescent fingerprint obtained from the reference sample containing a microbiota derived from a different environment A, B or C and a fluorescent fingerprint obtained from the test sample are compared. As a result, if the distance between the distributions of the fluorescent fingerprint obtained from the test sample and the fluorescent fingerprint obtained from the reference sample containing the microbiota derived from the environment A is stochastically most approximate, it is estimated or specified that the microbiota contained in the test sample is derived from the environment A.

According to a specific embodiment, the types and/or amounts of microbial strains contained in the microbiota can be determined. For example, a fluorescent fingerprint obtained from a fermented food sample containing a different strain A, B, or C (reference sample) and a fluorescent fingerprint obtained from a fermented food sample (test sample) are compared. When the distance between the distributions of the fluorescent fingerprint obtained from the fermented food sample containing unknown strains and the fingerprint obtained from the fermented food sample containing the strain A is stochastically approximate, it can be estimated or specified that the fermented food which is an analytical target contains the strain A.

According to a specific embodiment, the state and/or characteristics of the individual animal can be determined by using the test sample containing the microbiota derived from the individual animal. The types and/or amounts of the microorganisms that constitute the microbiota vary depending on the health condition and the constitution of the individual animal from which it is derived. In a specific embodiment, reference samples containing microbiota derived from animal individuals having different health conditions and constitutions are therefore preferably used.

In a specific embodiment, the reference sample may contain a bacterial flora derived from, for example, an obese individual organism or a non-obese individual organism. When the distance between the distributions of the fluorescent fingerprint obtained from the test sample and the fluorescent fingerprint obtained from the reference sample containing the bacterial flora derived from the obese individual is stochastically approximate in comparison between the fluorescent fingerprints obtained from the reference sample and the test sample, it can be estimated or specified that a subject from whom the test sample is derived is an obese individual or an individual at a high risk thereof.

In a specific embodiment, the reference sample may contain a bacterial flora derived from, for example, a healthy individual or an individual contracting an infectious disease. When the distance between the distributions of the fluorescent fingerprint obtained from the test sample and the fluorescent fingerprint obtained from the reference sample containing the bacterial flora derived from the individual contracting the infectious disease is stochastically approximate in comparison between the fluorescent fingerprints obtained from the reference sample and the test sample, it can be estimated or specified that a subject from whom the test sample is derived is an individual infected with a pathogenic strain of an infectious disease or is an individual at high risk thereof.

The method of the present embodiment enables analyzing a microbiota using only one or a few types of probes without the need to identify and/or quantify bacterial species that constitutes the microbiota and without the need to consider the possibility that unknown microorganisms are present.

### EXAMPLES

The present invention will now be further described by Examples, but it is not limited to the following Examples.

### 1. Synthesis of Probes (Probes 1 to 12)

A probe group obtained by introducing tetraphenylethylene (TPE) into a block copolymer of polyethylene glycol and poly-L-lysine (PEG-b-PLL) was synthesized by the procedure shown below.

Polyethylene glycol-block-poly-L-lysine trifluoroacetate (PEG-b-PLL, Mw:17200) [number of ethylene glycol repeat units: 104, number of L-lysine trifluoroacetate repeat units: 52] was purchased from Alamanda Polymers. Fmoc-Pro-OPfp (sc-235199), Fmoc-Nle-OPfp (sc-319878), Fmoc-Phe-OPfp (sc-250014) and Fmoc-Leu-OPfp (sc-235192) were purchased from Santa Cruz Biotechnology, Inc. Succinic anhydride (239690), phthalic anhydride (230064), 2,3-pyrazine dicarboxylic anhydride (405019) were purchased from Sigma-Aldrich. 1-(4-bromophenyl)-1,2,2-triphenylethylene (B3634) was purchased from Tokyo Chemical Industry Co., Ltd. n-Butyl lithium (1.6 mol/L hexane solution) (020-19071), tetrahydrofuran (THF) (extremely dehydrated) (207-17905), sodium sulfate (197-03345), pentafluorophenyl trifluoroacetate (326-32881), 1H-pyrazole-1-carboxamidine hydrochloride (322-31881), methanol (131-01826), dimethyl sulfoxide (DMSO) (049-07213), N,N-dimethylformamide (DMF) (049-02914), piperidine (166-02773), N,N-diisopropylethylamine (DIEA) (059-05352), and triethylamine (208-02643) were purchased from FUJIFILM Wako Pure Chemical Corporation. Fmoc-(Gly)3-OH (R00132), Fmoc-Ala(4-Pyri)-OH (M00669), and Fmoc-DAP(Fmoc)-OH (L00797) were purchased from WATANABE CHEMICAL INDUSTRIES, LTD.

### (1-1) Synthesis of Pentafluorophenyl 4-(1,2,2-triphenylethenyl)benzoate (TPE-CO-OPfp)

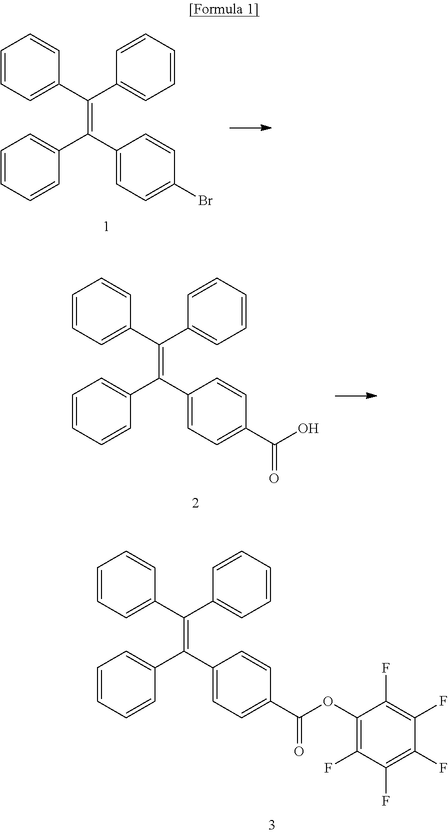 [SMILES: Brc1ccc(C(=C(c2ccccc2)c2ccccc2)c2ccccc2)cc1 ; O=C(O)c1ccc(C(=C(c2ccccc2)c2ccccc2)c2ccccc2)cc1 ; O=C(Oc1c(F)c(F)c(F)c(F)c1F)c1ccc(C(=C(c2ccccc2)c2ccccc2)c2ccccc2)cc1]

### (i) Synthesis of 4-(1,2,2-triphenylethenyl) benzoic acid (TPE-COOH) (Compound 2)

1-(4-bromophenyl)-1,2,2-triphenylethylene (Compound 1) (2.06 g, 5.0 mmol) was dissolved in tetrahydrofuran (70 mL) in an argon atmosphere, and the mixture was cooled to −78° C. n-butyl lithium (1.6 M hexane solution) (4.38 mL, 7.0 mmol) was slowly added dropwise to this solution over 5 minutes, and the mixture was stirred at −78° C. for 40 minutes. Crushed dry ice (15 g) was added to this reaction solution, and the mixture was stirred at −78° C. for 1 hour. After returning the reaction solution to room temperature and further stirring the reaction solution for 1 hour, hydrochloric acid (1.0 N, 50 mL) was then added, and the mixture was stirred at room temperature for 30 minutes. Then, ethyl acetate (100 mL) was added to the reaction solution to separate the aqueous layer. The obtained organic layer was washed with water (50 mL) once and with saturated saline solution (50 mL) once and dried over sodium sulfate. Then, the sodium sulfate was removed by filtration, and the solution was concentrated at reduced pressure. The residue was purified by silica gel column chromatography (elution solvent: ethyl acetate-chloroform) to obtain a Compound 2 (1.68 g, yield: 89%) as a white solid substance.

¹H NMR (500 MHz, CDCl₃) δ: 7.83 (d, 2H, ArH, J=8.5 Hz), 7.14-7.10 (m, 11H, ArH), 7.04-7.00 (m, 6H, ArH).

### (ii) Synthesis of Pentafluorophenyl 4-(1,2,2-triphenylethenyl)benzoate (TPE-CO-OPfp) (Compound 3)

The Compound 2 (750 mg, 2.0 mmol) was dissolved in DMF (30 mL) in an argon atmosphere. DIEA (0.68 mL, 4.0 mmol) and pentafluorophenyl trifluoroacetate (0.51 mL, 3.0 mmol) were added, and the mixture was stirred at room temperature for 100 minutes. Ethyl acetate (150 mL) was added to the reaction solution, and the obtained organic layer was washed with water (50 mL) 4 times and with a saturated saline solution (50 mL) once and dried over sodium sulfate. The sodium sulfate was removed by filtration, the solution was concentrated at reduced pressure, and the residue was purified by silica gel column chromatography (elution solvent: ethyl acetate hexane) to obtain a Compound 3 (980 mg, yield: 90%) as a white foamy substance.

¹H NMR (500 MHz, CDCl₃) δ: 7.92 (d, 2H, ArH, J=8.4 Hz), 7.20 (d, 2H, ArH, J=8.4 Hz), 7.16-7.11 (m, 9H, ArH), 7.06-7.01 (m, 6H, ArH).

### (1-2) Synthesis of Fmoc-(Gly)3-OPfp (Compound 5)

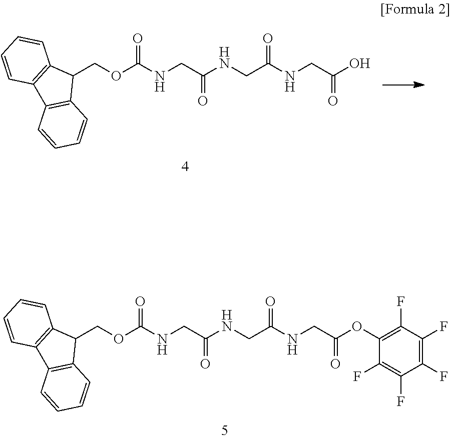 [SMILES: O=C(O)CNC(=O)CNC(=O)CNC(=O)OCC1c2ccccc2-c2ccccc21 ; O=C(CNC(=O)CNC(=O)OCC1c2ccccc2-c2ccccc21)NCC(=O)Oc1c(F)c(F)c(F)c(F)c1F]

Fmoc-(Gly)3-OH (Compound 4) (905 mg, 2.2 mmol) was dissolved in DMF (20 mL) in an argon atmosphere, DIEA (0.75 mL, 4.4 mmol) and pentafluorophenyl trifluoroacetate (0.56 mL, 3.3 mmol) were added, and the mixture was stirred at room temperature for 1 hour. Ethyl acetate (150 mL) was added to the reaction solution, the mixture was washed with water (60 ml) 4 times and with the saturated saline solution (60 mL) once, and the obtained organic layer was dried over sodium sulfate. The sodium sulfate was removed by filtration, and the solution was concentrated at reduced pressure. The obtained white solid was suspended in a mixed solution of ethyl acetate (15 mL) and hexane (45 mL), and the precipitate was filtered to obtain a Compound 5 (966 mg, yield: 76%) as a white powdery substance.

¹H NMR (500 MHz, DMSO-d₆) δ: 8.57 (t, 1H, NH, J=5.8 Hz), 8.21 (t, 1H, NH, J=5.8 Hz), 7.89 (d, 2H, ArH, J=7.5 Hz), 7.71 (d, 2H, ArH, J=7.5 Hz), 7.57 (t, 1H, NH, J=6.0 Hz), 7.42 (t, 2H, ArH, J=7.4 Hz), 7.33 (t, 2H, ArH, J=7.4 Hz), 4.33 (d, 2H, CH₂, J=5.8 Hz), 4.29 (d, 2H, CH₂, J=7.0 Hz), 4.23 (t, 1H, CH, J=7.0 Hz), 3.80 (d, 2H, CH₂, J=5.8 Hz), 3.68 (d, 2H, CH₂, J=6.0 Hz).

### (1-3) Synthesis of Fmoc-Ala(4-Pyri)-OPfp (Compound 7)

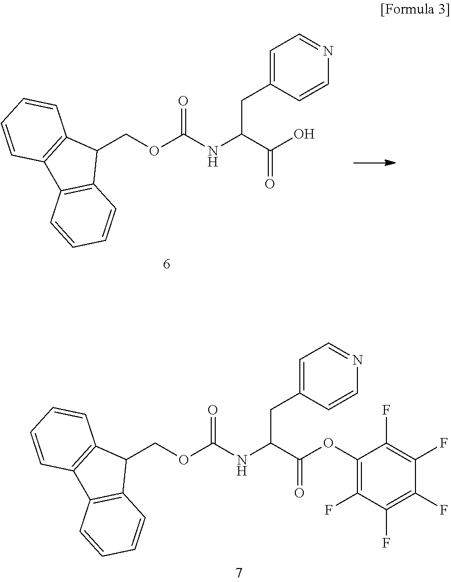 [SMILES: O=C(NC(Cc1ccncc1)C(=O)O)OCC1c2ccccc2-c2ccccc21 ; O=C(NC(Cc1ccncc1)C(=O)Oc1c(F)c(F)c(F)c(F)c1F)OCC1c2ccccc2-c2ccccc21]

Fmoc-Ala(4-Pyri)-OH (Compound 6) (1.55 g, 4.0 mmol) was dissolved in DMF (40 mL) in an argon atmosphere, DIEA (1.36 mL, 8.0 mmol) and pentafluorophenyl trifluoroacetate (1.02 mL, 6.0 mmol) were added, and the mixture was stirred at room temperature for 1.5 hours. Ethyl acetate (200 mL) was added to the reaction solution, the mixture was washed with water (80 ml) 4 times and with the saturated saline solution (80 mL) once, and the obtained organic layer was dried over sodium sulfate. The sodium sulfate was removed by filtration, and the solution was concentrated at reduced pressure. The residue was purified by silica gel column chromatography (elution solvent: ethyl acetate-hexane) to obtain a Compound 7 (1.86 g, yield: 84%) as a white solid substance.

¹H NMR (500 MHz, DMSO-d₆) δ: 8.49 (d, 2H, ArH, J=5.9 Hz), 8.26 (d, 1H, NH, J=7.8 Hz), 7.88 (d, 2H, ArH, J=7.6 Hz), 7.62 (dd, 2H, ArH, J=7.0, 5.2 Hz), 7.40 (m, 2H, ArH), 7.34 (d, 2H, ArH, J=5.9 Hz), 7.29 (m, 2H, ArH), 4.82 (ddd, 1H, CH, J=10.5, 7.8, 5.0 Hz), 4.39 (dd, 1H, CH₂a, J=10.7, 6.9 Hz), 4.30 (dd, 1H, CH₂b, J=10.7, 6.8 Hz). 4.20 (dd, 1H, CH, J=6.9, 6.8 Hz), 3.27 (dd, 1H, CH₂a, J=13.8, 5.0 Hz), 3.13 (dd, 1H, CH₂b, J=13.8, 10.5 Hz).

### (1-4) Synthesis of Fmoc-DAP(Fmoc)-OPfp (Compound 9)

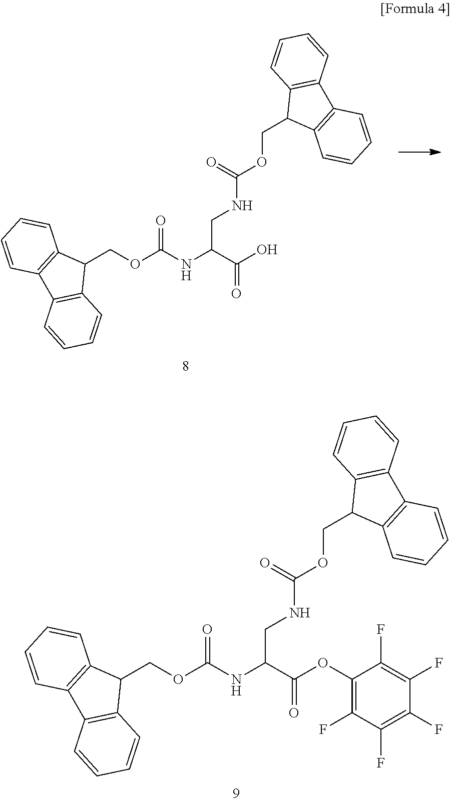 [SMILES: O=C(NCC(NC(=O)OCC1c2ccccc2-c2ccccc21)C(=O)O)OCC1c2ccccc2-c2ccccc21 ; O=C(NCC(NC(=O)OCC1c2ccccc2-c2ccccc21)C(=O)Oc1c(F)c(F)c(F)c(F)c1F)OCC1c2ccccc2-c2ccccc21]

Fmoc-DAP(Fmoc)-OH (Compound 8) (1.10 mg, 2.0 mmol) was dissolved in DMF (20 mL) in an argon atmosphere, DIEA (0.68 mL, 4.0 mmol) and pentafluorophenyl trifluoroacetate (0.51 mL, 3.0 mmol) were added, and the mixture was stirred at room temperature for 1 hour. Ethyl acetate (150 mL) was added to the reaction solution, the mixture was washed with water (60 mL) 4 times and with a saturated saline solution (60 mL) once, and the obtained organic layer was dried over sodium sulfate. The sodium sulfate was removed by filtration, and the solution was concentrated at reduced pressure. The obtained white solid was suspended in a mixed solvent of ethyl acetate (10 mL) and hexane (50 mL), and the precipitate was filtered to obtain a compound 9 (1.32 g, yield: 92%) as a white powdery substance.

¹H NMR (500 MHz, DMSO-d₆) δ: 8.10 (d, 1d, NH, J=7.7 Hz), 7.90-7.87 (m, 4H, Ar), 7.70-7.69 (m, 2H, Ar), 7.67-7.64 (m, 2H, ArH), 7.56 (t, 1H, NH, J=5.9 Hz), 7.42-7.39 (m, 4H, ArH), 7.32-7.27 (m, 4H, ArH), 4.64 (m, 1H, CH), 4.42-4.36 (m, 2H), 4.34-4.30 (m, 2H), 4.26-4.20 (m, 2H), 3.57-3.54 (m, 2H, CH₂).

### (1-5) Synthesis of PEG-b-PLL into which TPE Groups were Introduced (Probe 1)

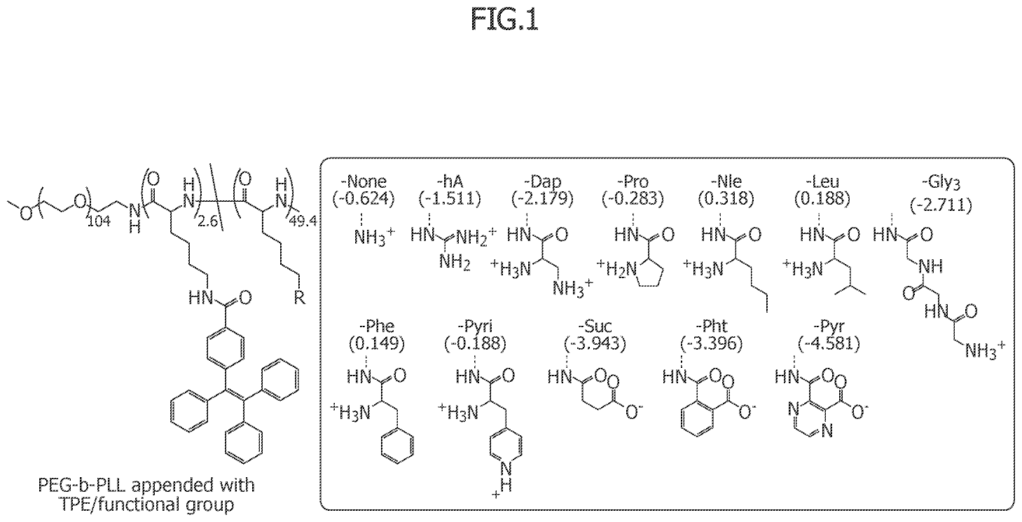

400 mg of PEG-b-PLL (23.3 μmol, number of moles of amino groups: 1200 μmol) was dissolved in 40 mL of DMF, 500 μL of triethylamine was added with stirring, and 1.51 mL of 40 mM TPE-CO-OPfp dissolved in DMF was further added. Then, the container was sealed and shielded from light in an argon atmosphere, and the mixture was stirred at room temperature for 24 hours. The mixture was dialyzed in sequence against ultrapure water once, methanol twice, ultrapure water once, 1 mM HCl twice, and ultrapure water once using a dialysis membrane (Spectra/Por6, (MWCO: 8 kDa)). Lyophilization was performed to obtain a powder of PEG-b-PLL into which TPE group was introduced (probe 1: -None (FIG. 1)).

It was confirmed by ¹H NMR (400 MHz, MeOD) from the area ratio of the peaks of 15 protons of a TPE except two protons of the phenyl ring next to the amide bond (δ=7.64 ppm) (δ=6.1 to 7.1 ppm) to the peak of the protons of α-CH in Lys side chains (δ=3.97 ppm) that 2.6 TPE groups were introduced into one molecule of PEG-b-PLL (data not shown).

### (1-6) Synthesis of PEG-b-PLL into which TPE Group and Functional Group were Introduced (Probes 2 to 12)

### (i) Introduction of Amino Acid

30 mg of the probe 1 (-None) (2.14 μmol, amino group: 106 μmol) was dissolved in 3 mL of DMSO, 54.4 μL of DIEA was added with stirring, and 2.65 mL of 200 mM Fmoc-various amino acids-OPfp dissolved in DMSO were further added. Then, the container was sealed and shielded from light in an argon atmosphere, and the mixture was stirred for 72 hours at room temperature. The mixture was dialyzed in sequence against ultrapure water once and methanol twice using a dialysis membrane (Spectra/Por6 (MWCO: 8 kDa)), and methanol was removed by an evaporator. 4 mL of DMSO was added, 1 mL of piperidine was added with stirring, and Fmoc groups were deprotected. Then, the container was sealed and shielded from light in an argon atmosphere, and the mixture was stirred for 40 hours at room temperature. The mixture was dialyzed in sequence against ultrapure water once, methanol three times, ultrapure water once, 1 mM HCl twice, and ultrapure water once using a dialysis membrane (Spectra/Por6 (MWCO: 8 kDa)). Lyophilization was performed to obtain powders of probes into which various amino acids were introduced. (probes 3 to 9: -Dap, -Pro, -Nle, -Leu, -Gly₃, -Phe, and -Pyri (FIG. 1))

It was confirmed from the disappearance of the peak of protons of εCH₂ in the Lys side chains (δ=2.97 ppm in MeOD, δ=3.04 ppm in D₂O) after the reaction in a ¹H-NMR chart that almost all the remaining Lys side chains were reacted (for -Phe, it was confirmed from the area ratio of the peak of five protons of the phenyl ring in the Phe side chain (δ=7.28 ppm) to the peaks of 15 protons in TPE except two protons of the phenyl ring next to the amide bond (δ=7.64 ppm) (δ=6.1 to 7.1 ppm) that the reaction proceeded completely) (data not shown).

(ii) Conversion into Guanidium Group

30 mg of the probe 1 (-None) (2.14 μmol, amino group: 106 μmol) is dissolved in 3.8 mL of methanol, 74.0 μL of triethyl amine was added with stirring, and 1.59 mL of 200 mM 1H-pyrazole-1-carboxamidine hydrochloride (PCA-Cl) dissolved in methanol was further added. Then, the container was sealed and shielded from light in an argon atmosphere, and the mixture was stirred for 24 hours at room temperature. The mixture was dialyzed in sequence against ultrapure water once, methanol twice, ultrapure water once, 1 mM HCl twice, ultrapure water once using a dialysis membrane (Spectra/Por6 (MWCO: 8 kDa)). Lyophilization was performed to obtain a powder of a probe in which amino groups were converted into guanidium groups (probe 2: -hA (FIG. 1)).

It was confirmed from the disappearance of the peak of the protons of εCH₂ in the Lys side chains (δ=2.97 ppm in MeOD) after the reaction in a ¹H-NMR chart that almost all the remaining Lys side chains were reacted (data not shown).

(iii) Carboxylation

30 mg of the probe 1 (-None) (2.14 μmol, amino group: 106 μmol) was dissolved in 3.8 mL of DMSO, 147.6 μL of triethyl amine was added with stirring, and 1.06 mL of 1 M various acid anhydrides dissolved in DMSO were further added. Then, the container was sealed and shielded from light in an argon atmosphere, and the mixture was stirred for 48 hours at room temperature. 2.5 mL of ultrapure water was added, and the mixture was stirred for 1 hour at room temperature. The mixture was dialyzed in sequence against 20% methanol once, ultrapure water once, 1 mM NaOH twice, and ultrapure water once using a dialysis membrane (Spectra/Por6 (MWCO: 8 kDa)).

Lyophilization was performed to obtain powders of probes in which amino groups were carboxylated (probes 10 to 12: -Suc, -Pht, -Pyr (FIG. 1)).

It was confirmed from the disappearance of the peak of the protons of εCH₂ in the Lys side chains (δ=2.97 ppm in MeOD, δ=2.77 ppm in DMSO-d₆) after the reaction in a ¹H-NMR chart that almost all the remaining Lys side chain were reacted (data not shown).

### 2. Culture of Intestinal Bacteria

Intestinal bacteria used in the present Examples are shown in Table 1. The intestinal bacterium strains were obtained from the culture collections of Japan Collection of Microorganisms (JCM) and Deutsche Sammlung von Mikroorganismen und Zellkulturen (DSMZ). The E. coli DH5a strain was purchased from GMbiolab Co., Ltd. The E. coli JM109 strain was purchased from Takara Bio Inc.

TABLE 1
Intestinal bacteria used in Examples

| Phylum | Genus | Species | Strain | Abbr. | Source |
|---|---|---|---|---|---|
| Firmicutes | Anaerostipes | coccoe | JCM13470T | F.A. | Human feces |
| Firmicutes | Blautia | hydrogenctrophica | DSM10507T | F.B. | Human feces |
| Firmicutes | Clostridium | citroniae | DSM19261T | F.C. | Clinical specimen |
| Firmicutes | Eubacterium | fissicatena | DSM3598T | F.E. | Goat gut |
| Firmicutes | Ruminococcus | gauvreauii | JCM14987T | F.R. | Human feces |
| Firmicutes | Lactococcus | lactis | JCM5805T | F.L.1 | unknown |
| Firmicutes | Lactobacillus | helveticus | JCM1004 | F.L.2 | Cheese |
| Bacteroidetes | Bacteroides | dorei | JCM13471T | B.B.1 | Human feces |
| Bacteroidetes | Bacteroides | coprophaus | JCM13818T | B.B.2 | Human feces |
| Bacteroidetes | Bacteroides | clarus | JCM16067T | B.B.3 | Human feces |
| Bacteroidetes | Bacteroides | oleiciplenus | JCM16102T | B.B.4 | Human feces |
| Actinobacteria | Bifidobacterium | longum | JCM12221 | A.B.1 | Intestine of infant |
| Actinobacteria | Bifidobacterium | thermophilum | JCM7033 | A.B.2 | Poultry feces |
| Actinobacteria | Bifidobacterium | faecale | 3CM7044 | A.B.3 | Human feces |
| Proteobacteria | Escherichia | coli | DH5α | P.E. 1 | GMbiolab |
| Proteobacteria | Escherichia | coli | JM109 | P.E. 2 | TaKaRa |

Preparation of culture medium: 41.7 g/L of modified GAM bouillon medium (NISSUI PHARMACEUTICAL CO., LTD.) was added to ultrapure water purged with N₂/CO₂ anaerobic mixed gas (80:20, v/v) and dissolved completely. The medium was dispensed to a 50-mL glass vial bottle, the liquid layer and the gas layer were purged sufficiently with N₂/CO₂ gas again, and the vial bottle was sealed with a butyl rubber stopper and an aluminum cap (NICHIDEN RIKA GLASS CO., LTD.). The vial bottle was autoclaved at 121° C. for 20 minutes and was stored at 4° C. until use.

Culture conditions: The intestinal bacterium strains were subjected to restoration according to respective instruction manuals, were inoculated into GAM medium and subjected to stationary culture at 37° C. The proliferation of bacteria was directly confirmed by the measurement of the turbidity of medium (OD₆₀₀) or microscope observation. Subsequently, the glycerol stock of each culture solution was prepared in the following procedure. An 80% glycerol solution was prepared in a 10-mL vial bottle, and the liquid layer and the gas layer were purged with N₂ gas. After autoclaving, 4 mL of each culture solution was added to 1 mL of this glycerol liquid (final glycerol concentration: 20%), and the glycerol stock was stored at −80° C. until use. The E. coli DH5a strain and the E. coli JM109 strain were inoculated into LB medium (1% tryptone, 0.5% yeast extract, 1% NaCl) and subjected to shaking culture at 37° C. Then, the glycerol stocks were prepared in the same procedure as the above and stored at −80° C.

### 3. Characterization of Probes

Probe solutions in which the probes 1 (-None) and 5 (-Nle) (final concentration: 180 nM) were dissolved in the following three types of solvents: (1) 24 mM MOPS (pH 7.0), (2)24 mM MOPS+180 mM NaCl (pH 7.0), or (3) 24 mM acetic acid+180 mM NaCl (pH 5.0) (all are final concentrations) were prepared. MOPS (M1254) was purchased from Sigma-Aldrich. Acetic acid (017-00256) was purchased from FUJIFILM Wako Pure Chemical Corporation. Each probe solution (100 uL) was added to a 96-well half area low adsorptive black microplate (Corning Incorporated, 3993). 20 μL of suspensions of bacteria (P. E. 1 or F. A.) at various concentrations in which the solvents were replaced with ultrapure water were further added. The mixtures were incubated at 35° C. for 10 minutes. Then, the fluorescence spectra were measured at an excitation wavelength of 330 nm and at emission wavelengths of 372 to 700 nm with a microplate reader (Cytation 5, BioTek Instruments, Inc.).

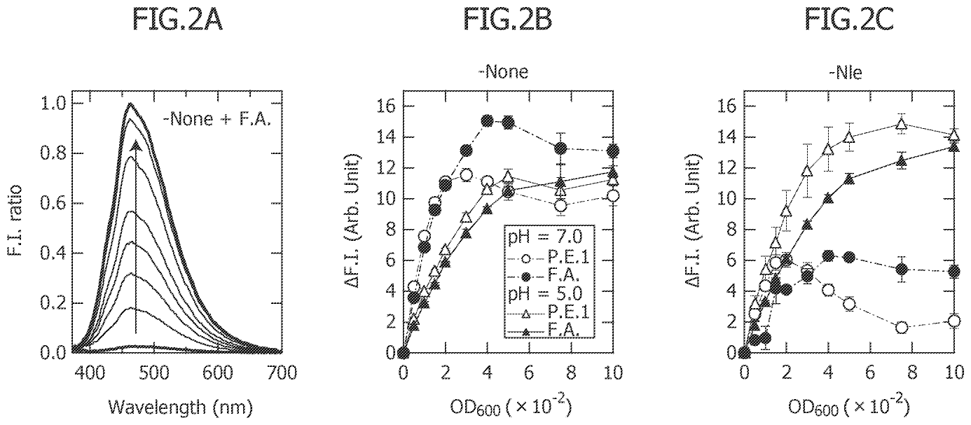

The results are shown in FIG. 2. FIG. 2(a) is atypical fluorescence spectrum of the probe 1 (-None)/the solvent (1) when bacteria (F. A.) were added at OD₆₀₀=0 to 0.10. The vertical axis (F. I. ratio) represents the fluorescence intensities with the fluorescence intensities at excitation wavelength (nm)=330 nm and at emission wavelength (nm)=460 nm defined as 1 when bacteria (F. A.) were added at OD₆₀₀=0.05. As the concentration of bacteria increased, the fluorescence intensity increased around 40-fold at the maximum. FIG. 2(b) shows the fluorescence intensity change in the probe solution of the probe 1 (-None)/the solvent (2) or (3) depending on the concentration of bacteria (emission wavelength: 460 nm). FIG. 2(C) shows the fluorescence intensity change in the probe solution of the probe 5 (-Nle)/the solvent (2) or (3) depending on the concentration of bacteria (emission wavelength: 460 nm). The vertical axis (AF. I.) represents the difference between the fluorescence intensity of the sample to which bacteria are not added and the fluorescence intensity of the sample to which bacteria are added. The fluorescence intensity change unique to each was seen depending on the type of the probe, the type of solvent, and the kind of bacteria. The results indicated the possibility that the cationic polymer TPE probes could identify the kind and/or concentration of bacteria.

### 4. Discrimination of Kinds of Intestinal Bacteria

12 μL of solutions of the probes 1 to 12 (1500 nM/ultrapure water) and 96 μL of a solvent (4): 25 mM MOPS+187.5 mM NaCl (pH 7.0) or a solvent (5): 25 mM acetic acid+187.5 mM NaCl (pH 5.0) were added to a 96-well half area low adsorptive black microplate using an automatic dispenser (PipetmaX, Gilson Incorporated). The mixtures were incubated at 35° C. for 10 minutes. Then, the fluorescence intensity (I₀) was measured with a microplate reader at the following two sets of excitation wavelengths (nm)/emission wavelengths (nm): (Ch1) 330/480 and (Ch2) 360/530. Subsequently, 12 μL of suspensions of the bacteria (Table 1, 16 types) (OD₆₀₀=0.40) in which the solvents were replaced with ultrapure water were added to the microplate, and the mixtures were incubated at 35° C. for 10 minutes. The final concentrations are as follows. Probe: 150 nM, solvent: 20 mM MOPS or acetic acid+150 mM NaCl, bacteria: OD₆₀₀=0.04. Then, the fluorescence intensity (I) was measured with a microplate reader at the following two sets of excitation wavelengths (nm)/emission wavelengths (nm): (Ch1) 330/480 and (Ch2) 360/530. 11 repeated measurements were performed on each mixed solution.

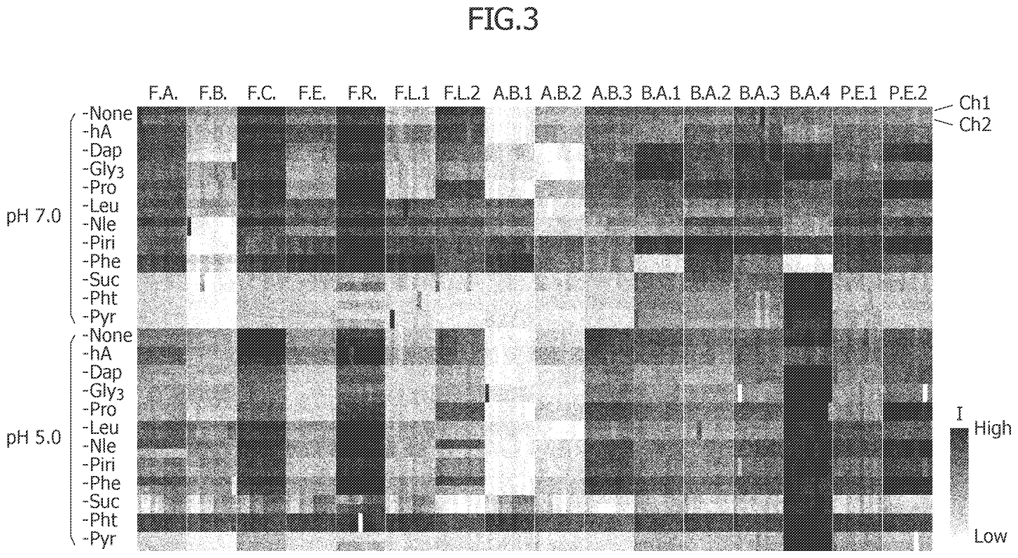

The heat map of the measurement results (16 bacteria×2 solvent conditions×12 probes×2 wavelength sets×11 measurements) is shown in FIG. 3. The fluorescence intensities of various probes were different depending on the kind of bacteria or solvent conditions, and a fluorescent fingerprint unique to each different bacterium was obtained.

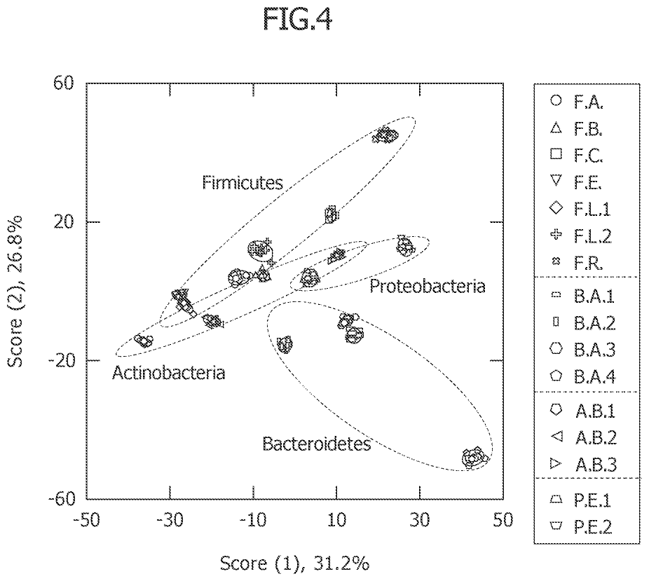

The results obtained by analyzing the abovementioned measurement results by linear discriminant analysis and plotting the resulting quadratic discriminant scores are shown in FIG. 4. The clusters of bacteria were distributed without being overlapped. Furthermore, when this result was analyzed by the jackknife method and the holdout method (4 measurement results selected at random were used as data for examination), bacteria could be identified with an accuracy of 100%. The above results demonstrated that bacteria could be distinguished at the species level using cationic polymer-environment-sensitive fluorophore probes. FIG. 4 also demonstrated that the metaclusters corresponding to bacterial phyla (shown with dashed lines) existed. The results demonstrated that bacteria could not only be distinguished at the species level, but also be distinguished at the phylum level using cationic polymer-TPE probes.

In the linear discriminant analysis of the abovementioned measurement results, the results when the fluorescence intensity (I) was not used, but the amount of change from the fluorescence intensity before adding bacteria to the fluorescence intensity after adding bacteria (I−I₀) was used are shown in FIG. 5. When I−I₀ was used, the clusters of some bacteria were overlapped. When the results were analyzed by the jackknife method, the discrimination accuracy was 99%. It was conjectured that it was because since the background fluorescence of the cationic polymer-TPE probes was very low, the disadvantages due to the measurement errors of I₀ exceeded the advantages of normalizing the measurement values by I₀. The above results demonstrated that when the cationic polymer-TPE probes were used, analysis with high accuracy is possible by only the measurement of the fluorescence intensity after adding bacteria.

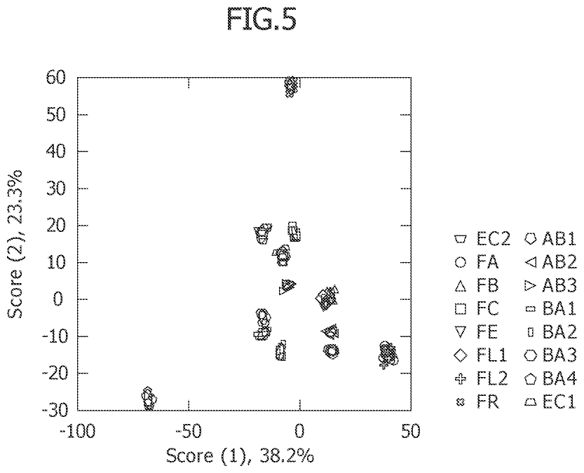

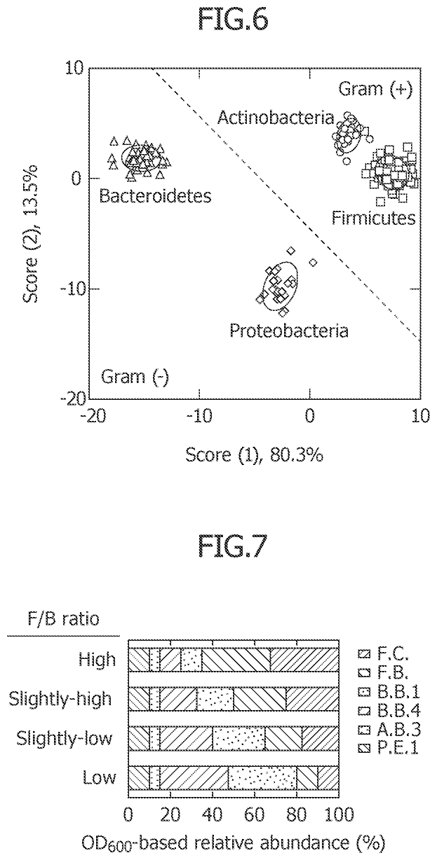

The results obtained by changing the labels of the bacteria from species to phyla (for example, both F. C. and F. E. are labeled merely as “Firmicutes”.) for analysis by linear discriminant analysis and plotting the resulting quadratic discriminant scores are shown in FIG. 6. The clusters of phyla were distributed without being overlapped. When the results were analyzed by the jackknife method, the discrimination accuracy was 99%. FIG. 6 demonstrated that gram-positive bacteria and gram-negative bacteria could also be separated (the upper right than the dashed line is gram-positive bacteria, and the lower left is gram-negative bacteria). Although it is not desired to adhere to specific theory, the results demonstrate the possibility that the difference in the cell wall structure of bacteria (peptidoglycan layer) has been recognized.

### 5. Discrimination of Obesity Model Microbiota Samples

Four types of obesity model microbiota samples containing six types of bacteria at different ratios (OD₆₀₀=0.40) were prepared (FIG. 7) based on the knowledge that the ratio of bacteria belonging to Firmicutes to bacteria belonging to Bacteroides in a human gut microbiota (F/B ratio) correlates with obesity (BMC Microbiol., 2017, 17:120). The fluorescence intensity was measured according to the same procedure and conditions as the abovementioned 4 using six types of probes (probes 1, 3, 6, 7, 8, and 11: -None, -Dap, Gly₃, -Leu, -Phe, and -Pht). 22 repeated measurements were performed on each sample.

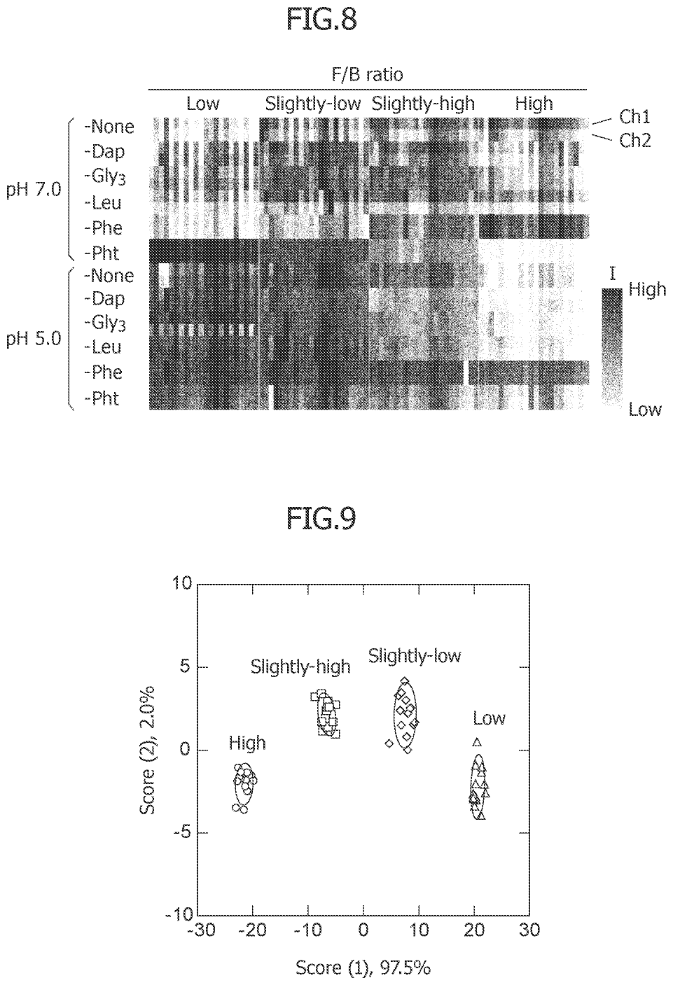

The heat map of the measurement results (4 samples×2 solvent conditions×6 probes×2 wavelength sets×22 measurements) is shown in FIG. 8. It was demonstrated that a fluorescent fingerprint unique to every sample was obtained.

The results obtained by analyzing the results by linear discriminant analysis and plotting the resulting quadratic discriminant scores are shown in FIG. 9. The clusters of model microbiota samples were distributed without being overlapped. When the results were analyzed by the jackknife method and the holdout method (8 measurement results selected at random were used as data for examination), samples could be identified with accuracies of 100% and 97% (31 of 32 measurement results were correct), respectively. The above results indicated the possibility that gut microbiota derived from individuals differing in the state of obesity could be distinguished using cationic polymer-environment-sensitive fluorophore probes.

### 6. Discrimination of Gut Microbiota Samples of Sleep Disorder Model Mice

### (6-1) Production of Sleep Disorder Model Mice

Sleep disorder model mice were produced based on a past report of the present inventors (PLOS ONE, 2013, 8:e55452; Neurosci. Lett., 2017, 653-362). C3H—HeN mice (male, 8-weeks-old, eight in total) were divided into two groups (four: having no sleep disorder stress (control group) and four: having sleep disorder stress (stress group)), reared in running wheel cages (SW-15; Melquest Ltd.), and freely provided with normal food and water over 1 to 2 weeks. The mice were subjected to acclimatization raising for 10 days under conditions of 22° C., humidity: 50%, light period: dark period=12 hours:12 hours (−10th to the 0th days). Then, the control group was continuously reared under the same conditions, and the stress group was continuously reared under the same conditions except that the stress group was moved to cages for producing sleep disorder model mice (SW-15-SD, Melquest Ltd.) (0th to 28th days). As action rhythm data, the running wheel activity was measured every minute with a Chronobiology Kit (Stanford Software Systems).

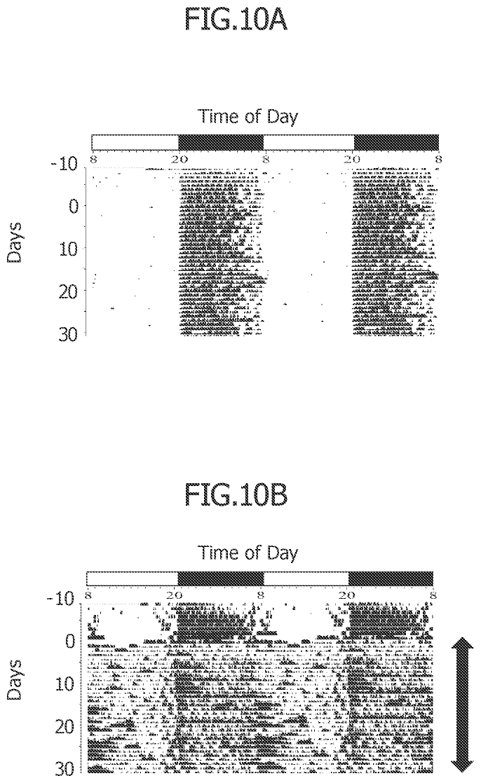

The results are shown in FIG. 10. Although both the control group and the stress group were more active in the dark period (night) than in the light period (daytime) during the acclimatization raising period, the stress group changed so as to be active at random regardless of whether in the light period or in the dark period due to sleep disorder stress (FIG. 10(b)).

### (6-2) Analysis of Mouse Feces Samples

28 days after, the mice were moved to sterilized new cages, excreted feces were promptly collected in a microtube and frozen in liquid nitrogen. Then, the feces were stored at −80° C. until analysis. Gut microbiota samples were prepared from frozen feces by a method based on a report of Benno et al. (Sci. Rep., 2011, 2:233). Each feces sample was weighed, and phosphate buffered saline (PBS) was added to obtain a 40 mg/mL suspension. For this suspension, the process of mixing for 1 minute and leaving to stand for 5 minutes at 4° C. was repeated multiple times. Then, the mixture was centrifuged at 8000 g and at 4° C. for 10 minutes, and the supernatant was removed. The obtained pellet was suspended in PBS, the mixture was centrifuged at 8000 g and at 4° C. for 10 minutes again, and the supernatant was removed. The obtained pellet was suspended in PBS, and the suspension filtered through a pluriStrainer™ (mesh size: 40 μm, pluriSelect Life Science UG & Co. KG) was prepared as a gut microbiota sample (200 μg/mL feces in PBS). The fluorescence intensity of the obtained sample was measured according to the same procedure and conditions as the abovementioned 5. 11 repeated measurements were performed on each sample.

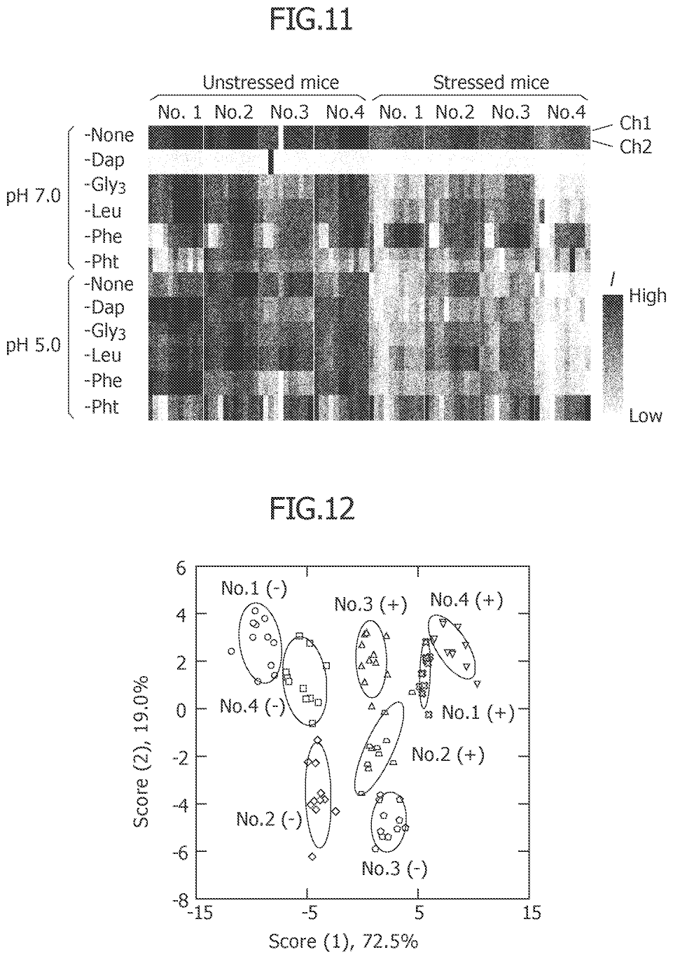

The heat map of the measurement results (8 samples×2 solvent conditions×6 probes×2 wavelength sets×11 measurements) is shown in FIG. 11. It was demonstrated that a fluorescent fingerprint unique to every sample was obtained.

The results obtained by analyzing the results by linear discriminant analysis and plotting the resulting quadratic discriminant scores are shown in FIG. 12. The clusters of samples were distributed without being overlapped (discrimination accuracy by the jackknife method: 90%).

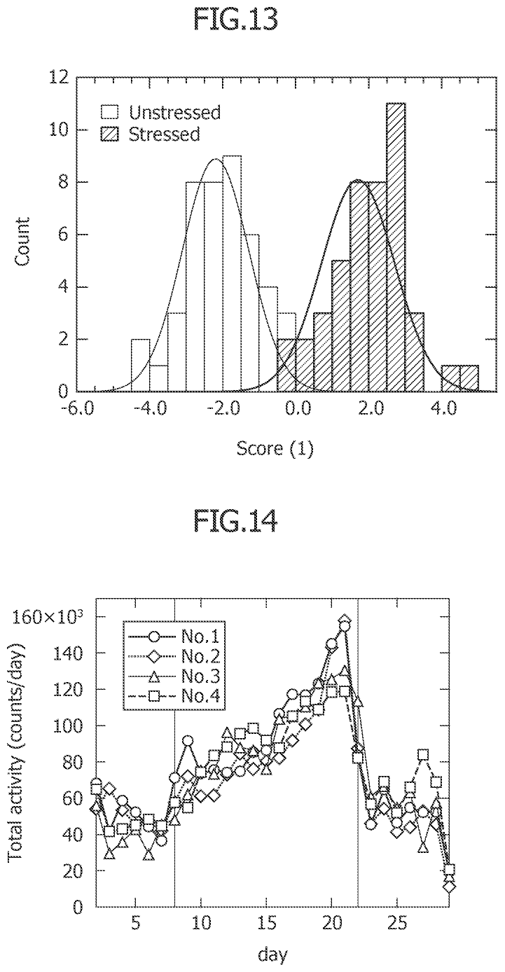

Furthermore, the results obtained by changing the labels of the samples depending on whether the mice were subjected to sleep disorder stress or not (Stressed/Unstressed) in the above measurement results for analysis by linear discriminant analysis again are shown in FIG. 13. It was demonstrated that although the results of the control group and the stress group were slightly overlapped, there was a clear difference (Student's t-test, p<0.003). When the results were analyzed by the jackknife method and the holdout method (16 measurement results selected at random were used as the data for examination), the control group and the stress group could be identified with accuracies of 91% and 94% (30 of 32 measurement results were correct), respectively. The above results demonstrated that the state of an individual animal could be determined by analyzing a gut microbiota sample using cationic polymer-environment-sensitive fluorophore probes.

### 7. Discrimination of Gut Microbiota Samples of Model Mice of Insufficient Exercise

To investigate whether or not still smaller changes in the gut microbiota than changes due to sleep disorder could be detected, model mice with insufficient exercise were produced. C3H—HeN mice (male, 6-weeks-old (around 40-days-old), four in total) were freely provided with normal food and water in running wheel cages (SW-15, Melquest Ltd.) under conditions of 22° C., humidity: 50%, and light period:dark period=12 hours:12 hours and subjected to acclimatization raising over 2 weeks. Then, an exercise quantity meter (nano tag, KISSEI COMTEC CO., LTD.) was implanted in the abdomen of each mouse (the −34th day), and each mouse was moved to an individual running wheel cage (the −24th day) and was reared with the running wheel unfixed (until the 0th day). Then, each mouse was reared with the running wheel fixed for 1 week (the 1st to the 8th days), was reared with the running wheel unfixed again (the 9th to the 21st days), and was then reared with the running wheel fixed for 1 week (the 22nd to the 29th days). The integrated values of the number of vibrations measured by the exercise quantity meter were obtained as the action data of the mouse.

The results are shown in FIG. 14. While the exercise quantity was 40,000 to 60,000 counts/day in the first period in which the running wheel was fixed, the exercise quantity increased gradually, and were 120,000 to 140,000 counts/day on the 20th day in the period in which the running wheel was unfixed. Then, the exercise quantity returned to 40,000 to 60,000 counts/day in the period in which the running wheel was fixed again.

The excreted feces were promptly collected in microtubes 12 days after the running wheel was unfixed (on the 20th day) and 5 days after the running wheel was fixed (on the 27th day), frozen in liquid nitrogen, and stored at −30° C. until analysis. The gut microbiota samples were prepared from the frozen feces by the same procedure as the abovementioned (6-2). The fluorescence intensity of each of the obtained samples was measured according to the same procedure and conditions as the abovementioned 5. Six repeated measurements were performed on each sample.

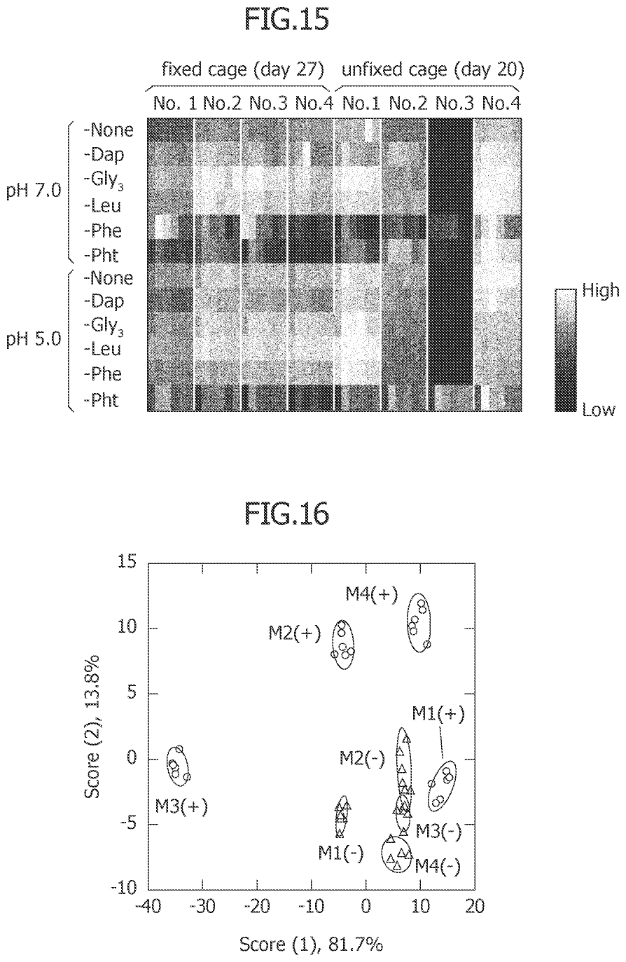

The heat map of the measurement results (8 samples×2 solvent conditions×6 probes×2 wavelength sets×6 measurements) is shown in FIG. 15. It was demonstrated that a fluorescent fingerprint unique to every sample was obtained.

The results obtained by analyzing the results by linear discriminant analysis and plotting the resulting quadratic discriminant scores are shown in FIG. 16. The clusters of the samples were distributed without being overlapped. When the results were analyzed by the jackknife method and the holdout method (16 measurement results selected at random were used as the data for examination), the individual mice and the periods thereof in which the running wheel was fixed and in which the running wheel was unfixed could be identified with accuracies of 96% and 94% (15 of 16 measurement results were correct).

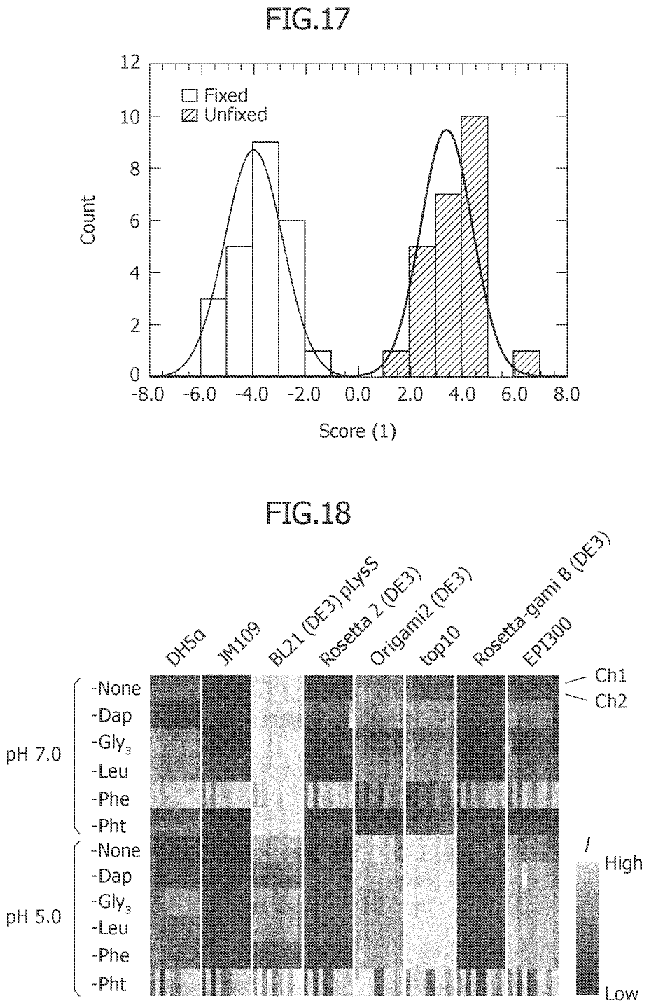

The results obtained by changing the labels of the samples depending on whether exercise was restricted or not (whether the running wheel was fixed or unfixed) (fix/unfix) and analyzing the abovementioned results by linear discriminant analysis are shown in FIG. 17. It was demonstrated that the group not restricted in exercise and the group restricted in exercise did not overlap, and there was a clear difference therebetween. When the results were analyzed by the jackknife method and the holdout method (16 measurement results selected at random were used as the data for examination), the mice could be identified by both with an accuracy of 100% depending on whether the mice were restricted in exercise or not. The above results demonstrated that whether an individual animal had the habit of exercising or not could be determined by analyzing a gut microbiota sample using cationic polymer-environment-sensitive fluorophore probes.

### 8. Discrimination of Intestinal Bacterium Strains

As showed in the abovementioned 4, various intestinal bacteria can be identified, not only at the “species” level, but also at the “phylum” level, using cationic polymer-environment-sensitive fluorophore probes. In the case of bacteria, the “strain”, which is a still lower class than these levels, however exists. Since different bacterial strains separated from the same bacterial species has the same genetic traits in principle, it is difficult to identify them by presently general gene analysis techniques such as 16S rRNA analysis. Meanwhile, patent infringement by the unauthorized use of patented bacterial strains, or the like, has become a problem, and a method for discriminating the compositions of microbiota at the strain level has been desired in recent years. Accordingly, in the present Example, whether different strains separated from the same bacterial species could be identified using cationic polymer-environment-sensitive fluorophore probes was tested. E. coli strains used in the present Example are shown in Table 2.

TABLE 2
Intestinal bacteria used in Examples

| Phylum | Genus | Species | Strain | Source |
|---|---|---|---|---|
| Proteobacteria | Escherichia | coli | DH5α | GMbiolab |
| Proteobacteria | Escherichia | coli | JM109 | TaKaRa |
| Proteobacteria | Escherichia | coli | BL21 (DE3) | BioDynamics |
| | | | pLysS | |
| Proteobacteria | Escherichia | coli | Rosetta 2 (DE3) | Novagen |
| Proteobacteria | Escherichia | coli | Origami 2 (DE3) | Novagen |
| Proteobacteria | Escherichia | coli | top10 | Invitrogen |
| Proteobacteria | Escherichia | coli | Rosetta-gami B | Novagen |
| | | | (DE3) | |
| Proteobacteria | Escherichia | coli | EPI300 | Epicentre |

Culture conditions: E. coli strains were inoculated into LB medium (1% tryptone, 0.5% yeast extract, and 1% NaCl) and subjected to shaking culture at 37° C. Then, 4 mL of each culture solution was added to 1 mL of 80% autoclaved glycerol solution (final glycerol concentration: 20%), and the strain was stored at −80° C. until use.

The fluorescence intensity was measured according to the same procedure and conditions as the abovementioned 4 using six types of probe solutions (probes 1, 3, 6, 7, 8, and 11: -None, -Dap, -Gly₃, -Leu, -Phe, -Pht, 1500 nM/ultrapure water) and E. coli suspensions replaced with ultrapure water (OD₆₀₀=0.40).

The heat map of the measurement results of the amount of change from the fluorescence intensity before adding E. coli to the fluorescence intensity after adding E. coli (I−I₀) (8 types of E. coli×2 solvent conditions×6 probes×2 wavelength sets×11 measurements) is shown in FIG. 18. The fluorescence intensities of various probes are different depending on the strain of E. coli and the solvent conditions, and fluorescent fingerprints unique to the different strains were obtained.

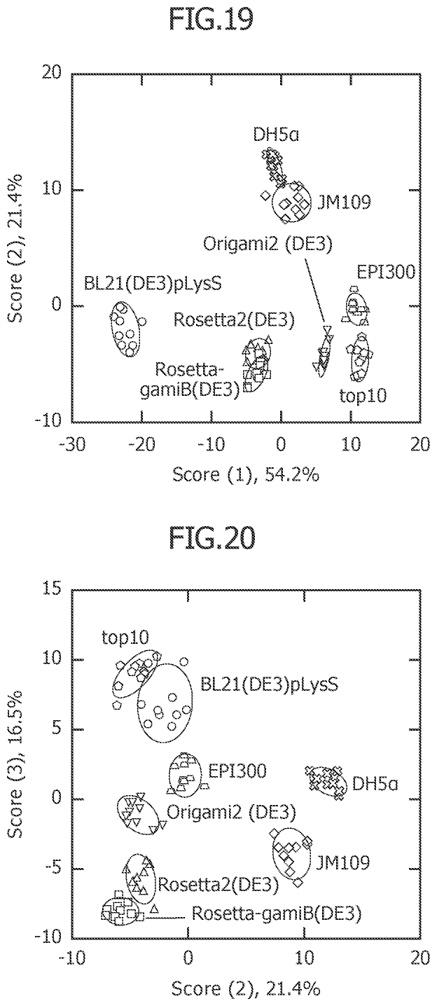

The results obtained by analyzing the abovementioned results by linear discriminant analysis and plotting the resulting linear discriminant scores and quadratic discriminant scores are shown in FIG. 19. The results obtained by analyzing the abovementioned results by linear discriminant analysis and plotting the resulting quadratic discriminant scores and cubic discriminant scores are shown in FIG. 20. The clusters of the strains were distributed on the plot of the first discriminant scores and the second discriminant scores without being overlapped except the Rosetta-Gami B (DE3) strain and the Rosetta 2 (DE3) strain. The cluster of the Rosetta-Gami B (DE3) strain and Rosetta 2 (DE3) strain were distributed on the plot of the second discriminant scores and the third discriminant scores without overlapping. When the results were analyzed by the jackknife method, bacteria could be identified with an accuracy of 99%. The above results demonstrated that bacteria could also be distinguished at the strain level using cationic polymer-environment-sensitive fluorophore probes.

### 9. Synthesis of Probes (Probes 13-17)

The probe group obtained by introducing fluorescein isothiocyanate (FITC) into a block copolymer of polyethylene glycol and poly-L-lysine (PEG-b-PLL) was synthesized by the procedure shown below.

### (9-1) Synthesis of PEG-b-PLL into which FITC Groups were Introduced (Probe 13)

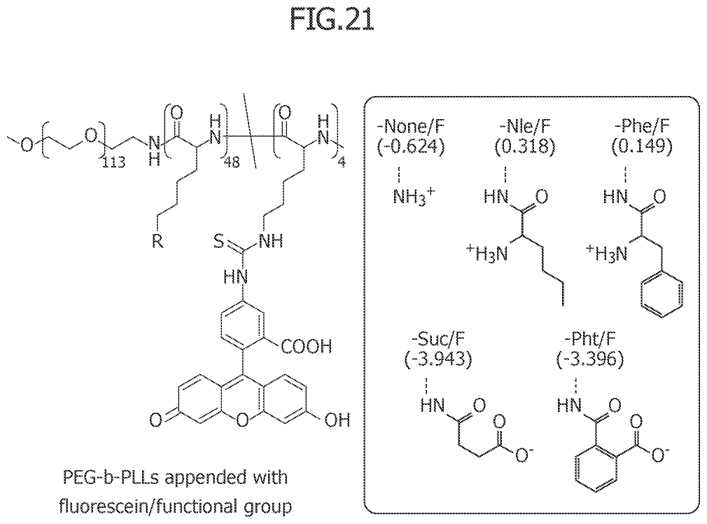

200 mg PEG-b-PLL (11.6 μmol, number of moles of amino groups: 600 μmol) is dissolved in 10 mL of methanol, 250 μL of triethyl amine was added with stirring, and 7.6 mL of 8 mM of FITC (F007, DOJINDO LABORATORIES) dissolved in methanol was further added. Then, the container was sealed and shielded from light in an argon atmosphere, and the mixture was stirred for 24 hours at room temperature. The mixture was dialyzed in sequence against ultrapure water once, methanol twice, ultrapure water once, 1 mM HCl twice, and ultrapure water once using a dialysis membrane (Spectra/Por6 (MWCO:8 kDa)). Lyophilization was performed to obtain a powder of PEG-b-PLL into which FITC groups were introduced (probe 13: -None/F (FIG. 21)). It was confirmed from the absorbance at 495 nm in 10 mM NaOH that 3.9 FITC groups were introduced into one molecule of PEG-b-PLL (data not shown).

### (9-2) Synthesis of PEG-b-PLL into which FITC Groups and Functional Groups were Introduced (Probes 14 to 17)

The same procedure was performed as the above (1-6) to obtain powders of PEG-b-PLL into which FITC groups and functional groups were introduced (probe 14 to 17: -Nle/F, -Phe/F, -Suc/F, and Pht/F (FIG. 21)) except that the probe 13 (-None/F) was used instead of the probe 1 (-None).

### 10. Preparation of Indigo Dyeing Bacterium Flora Samples

Indigo dyeing bacterium florae were prepared by the method into which a past report of the present inventors was modified (World J. Microbiol. Biotechnol., 2017, 33:70). Composted material of indigo leaves (sukumo, prepared indigo leaves) and extracted liquid of wood ash (lye) were mixed in a vat to prepare an indigo fermented liquid containing an indigo dyeing bacterium flora (pH 11.2). The sukumo, prepared indigo leaves, was purchased from Aikuma Senryo (03250503). Wood ash was mixed with water and boiled for 10 minutes to prepare the lye. The indigo fermented liquid was left to stand at 26° C. and stirred with a stirring rod once a day. The pH of the indigo fermented liquid was adjusted to the range of 10.3 to 11.3 by adding calcium hydroxide.

Indigo fermented liquid was collected in a microtube in every certain period of time, and glycerol was added so that the concentration was 25% to prepare an indigo dyeing bacterium flora suspension for freeze storage at −20° C. until analysis. The suspension was thawed at 4° C., filtered through a pluriStrainer™ (mesh size: 40 μm, pluriSelect Life Science UG & Co. KG), and further filtered through a pluriStrainer™ (mesh size: 10 μm, pluriSelect Life Science UG & Co. KG). The obtained filtrate was centrifuged at 8000 G and at 4° C. for 10 minutes, and the supernatant was removed. The obtained pellet was suspended in 10 mM MOPS (pH 7.0)+100 mM NaCl, the suspension was centrifuged at 8000 G and at 4° C. for 10 minutes, and the supernatant was removed. This step was repeated further twice. The obtained pellet suspended in 10 mM MOPS (pH 7.0)+100 mM NaCl was used as an indigo dyeing bacterium flora sample.

### 11. Characterization of Probes

Probe solutions in which probes 13 (-None/F) and 14 (-Nle/F) (final concentration: 75 nM) were dissolved in 20 mM MOPS (pH 7.0)+150 mM NaCl (final concentration) were prepared. The probe solutions (100 μL) were added to a 96-well half area low adsorptive black microplate (Corning Incorporated, 3993), 20 μL of indigo dyeing bacterium flora samples (prepared from indigo fermented liquids as of the 84th day or the 280th day) at various concentrations in which the solvents were replaced with 10 mM MOPS (pH 7.0)+100 mM NaCl were further added, and the mixtures were incubated at 35° C. for 10 minutes. Then, the fluorescence spectrum was measured with a microplate reader (Cytation 5, BioTek Instruments, Inc.) at an excitation wavelength of 460 nm and at an emission wavelength of 501 to 700 nm.

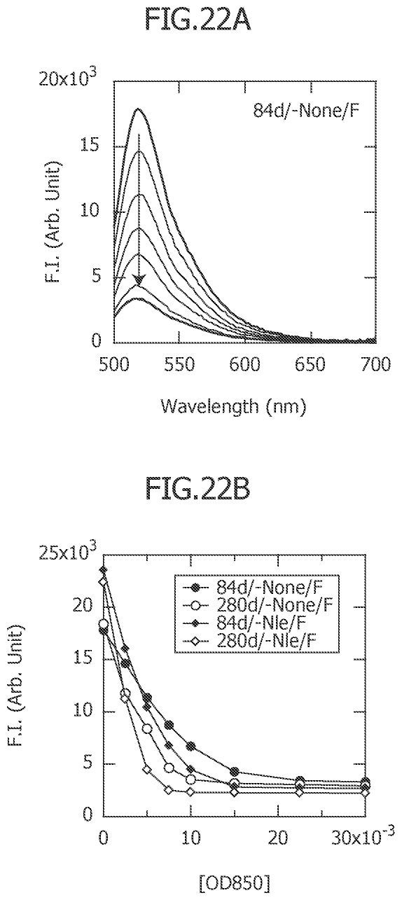

The results are shown in FIG. 22. FIG. 22(a) is atypical fluorescence spectrum of the probe 13 (-None/F) when an indigo dyeing bacterium flora sample (prepared from indigo fermented liquid as of the 84th day) was added at OD₈₅₀=0 to 0.03. As the concentration of indigo dyeing bacteria increased, the fluorescence intensity decreased to around 19% at the maximum. FIG. 22(b) illustrates the fluorescence intensity changes of the probe solutions of the probe 13 (-None/F) and the probe 14 (-Nle/F) depending on the concentration of indigo dyeing bacteria (emission wavelength: 521 nm). In the figure, “84d” represent an indigo dyeing bacterium flora sample prepared from indigo fermented liquid as of the 84th day, and “280d” represent an indigo dyeing bacterium flora sample prepared from indigo fermented liquid as of the 280th day. The fluorescence intensity change unique to each was seen depending on the type of the probe and the state of indigo fermented liquid. The results indicated the possibility that cationic polymer-FITC probes could distinguish indigo fermented liquids different in the state.

### 12. Discrimination of Indigo Fermented Liquids

In indigo dyeing, the dyeability such as the shade or chromaticness of color changes depend on the composition of an indigo dyeing bacterium flora in indigo fermented liquid. Therefore, it has been required that the state of the indigo fermented liquid is ascertained, and it is promptly determined whether to promote or stop fermentation. In the present situation, such determination is, however, made based on the appearance and the smell of fermented liquid, and depends on the experience and intuition of craftsmen. Accordingly, in the present Example, whether the dyeability of indigo fermented liquid could be distinguished using cationic polymer-environment-sensitive fluorophore probes or not was tested.

### (12-1) Evaluation of Dyeability of Indigo Fermented Liquid

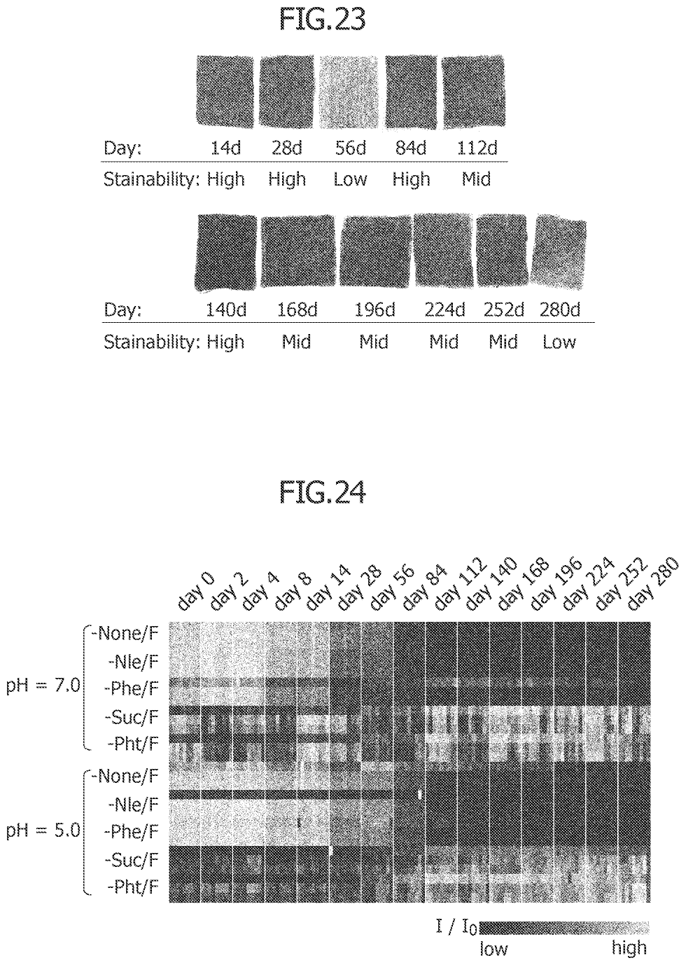

Small pieces of a cotton fabric were dipped in fermented liquid for 30 seconds, and the dyeing intensities were classified into three ranks (high/mid/low) visually. The results are shown in FIG. 23. Since the cotton pieces were hardly dyed with the indigo fermented liquids as of the 0th day, the 2nd day, the 4th day, and the 8th day, these were all classified into “low” (data not shown).

### (12-2) Analysis of Indigo Dyeing Bacterium Flora Sample

10 μL of probe 13 to 17 solutions (750 nM/ultrapure water) and 80 μL of a solvent (4) 25 mM MOPS+187.5 mM NaCl (pH 7.0) or a solvent (5) 25 mM acetic acid+187.5 mM NaCl (pH 5.0) were added to a 96-well half area low adsorptive black microplate using an automatic dispenser (pipetmaX, Gilson Incorporated), and each of the mixtures was incubated at 35° C. for 10 minutes. Then, the fluorescence intensity (I₀) was measured with a microplate reader at the following three sets of excitation wavelengths (nm)/emission wavelengths (nm): (Ch 1) 350/520, (Ch 2) 470/520, and (Ch 3) 515/560. Then, 10 μL of the indigo dyeing bacterium flora samples (15 types) (OD₈₅₀=0.10) in which the solvents were replaced with 10 mM MOPS+100 mM NaCl were added to the microplate, and each of the mixtures was incubated at 35° C. for 10 minutes. The final concentration was as follows: probe: 75 nM, solvent: 20 mM MOPS or acetic acid+150 mM NaCl, and bacteria: OD₈₅₀=0.01. Then, the fluorescence intensity (I) was measured with the microplate reader at the following three sets of excitation wavelengths (nm)/emission wavelengths of (nm):(Ch1) 350/520, (Ch2) 470/520, and (Ch 3) 515/560. Nine repeated measurements were performed on each mixed solution.

The heat map of the measurement results (15 indigo dyeing bacterium florae×2 solvent conditions×5 probes×3 wavelength sets×9 measurements) is shown in FIG. 24. It was demonstrated that a fluorescent fingerprint unique to every indigo dyeing bacterium flora sample was obtained.

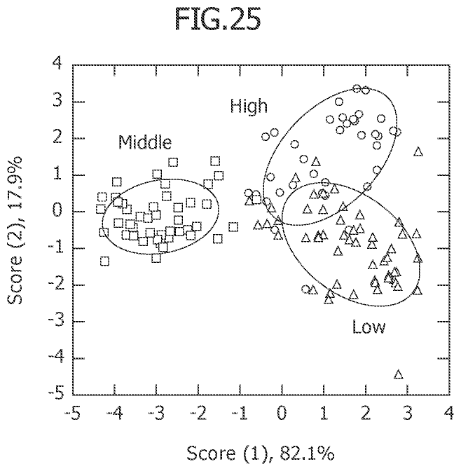

The results obtained by changing the label of each sample of the abovementioned measurement results into the dyeing intensity (high/middle/low), analyzing the results by linear discriminant analysis, and plotting the resulting quadratic discriminant scores are shown in FIG. 25. Although the clusters of “high” and “low” were seen to slightly overlap, the clusters of “middle” was distributed without overlapping with other clusters. When the results were further analyzed by the jackknife method, the dyeing intensity groups could be identified with an accuracy of 76%. The above results demonstrated that the dyeing intensity of an indigo dyeing bacterium flora could be distinguished using cationic polymer-environment-sensitive fluorophore probes.

1. A method for analyzing a microbiota, comprising:
(1) dissolving a probe capable of non-specifically interacting with a plurality of microorganisms in a plurality of solvents having different ionic strengths and pH levels,
wherein the probe comprises:
(a) a cationic polymer comprising at least five primary amino groups in one molecule and having a weight-average molecular weight of 1,000 to 500,000 and
(b) an environment-sensitive fluorophore,
wherein the fluorophore is covalently bonded to some of the primary amino groups in the cationic polymer;
(2) adding a test sample containing the microbiota to a plurality of probe solutions prepared in the step (1), and thereby microorganisms in the test sample and the probe are interacted non-specifically;
(3) measuring fluorescence intensities of the plurality of probe solutions to which the test sample has been added in the step (2); and
(4) comparing the pattern of fluorescence intensities obtained in the step (3) with the pattern of fluorescence intensities obtained from a reference sample.

2. The method according to claim 1, wherein the environment-sensitive fluorophore is an aggregation-induced emission fluorophore.

3. The method according to claim 1, wherein the aggregation-induced emission fluorophore is tetraphenylethylene or a derivative thereof.

4. The method according to claim 1, wherein the cationic polymer is a linear or branched polyamino acid, polyallylamine, polyamidoamine, or poly alkyleneimine.

5. The method according to claim 4, wherein the polyamino acid is polylysine or polyornithine.

6. The method according to claim 1, wherein the environment-sensitive fluorophore is covalently bonded to 1 to 50% of the primary amino groups in the cationic polymer.

7. The method according to claim 1, wherein a functional group selected from the group consisting of a guanidium group, a carboxyl group, and an amino acid is introduced into at least some of the primary amino groups not covalently bonded to the environment-sensitive fluorophore in the cationic polymer.

8. The method according to claim 1, wherein the measurement of the fluorescence intensities in the step (3) is performed at a plurality of excitation wavelengths and emission wavelengths.

9. The method according to claim 1, wherein types and/or amounts of microbial strains comprised in the microbiota are determined by the step (4).

10. The method according to claim 1, wherein the microbiota is derived from an individual animal, and a state and/or a characteristic of the individual animal is determined by the step (4).

11. The method according to claim 10, wherein the individual animal is a human.
